(19) United States
(12) Patent Application Publication
(10) Pub. No.: US 2011/0059953 A1
(43) Pub. Date: Mar. 10, 2011

(54) COMPOUNDS FOR USE IN STABILIZING P53 MUTANTS

(76) Inventors: Frank Boeckler, Munich (DE); Andreas Joerger, Cambridge (GB); Alan Fersht, Cambridge (GB)

(21) Appl. No.: 12/991,153

(22) PCT Filed: May 7, 2009
(86) PCT No.: PCT/GB09/01160

§ 371 Date: Nov. 5, 2010

(30) Foreign Application Priority Data
May 7, 2008 (GB) 0808282.8

(51) Int. Cl. A61K 31/409; A61K 31/5377; A61K 31/438; A61K 31/4545; A61K 31/41; A61K 31/433; A61K 31/454; A61K 31/497; A61K 31/551; C07K 1/107; A61P 35/00; C12N 5/02; G01N 33/566

(52) U.S. Cl. 514/218; 514/411; 514/232.8; 514/278; 514/316; 514/381; 514/363; 514/323; 514/255.05; 530/408; 530/409; 435/375; 436/501

(57) ABSTRACT

Compounds of formula (I): wherein X is selected from CR^{X} and N; R^{N1} is selected from H and C₁₋₄ alkyl, which may be substituted by SH or halo; R^{G1} is selected from H and SH; R^{C2} is selected from H and optionally substituted C₁₋₇ alkyl; R^{C3} is selected from H and optionally substituted C₁₋₇ alkyl; R^{x} is selected from H, OH and NH₂; R^{C4} is selected from: (i) an optionally substituted C₃₋₁₂ N-containing heterocyclyl; (ii) C(═O)NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl or RN5 and RN6 and the nitrogen atom to which they are attached form an optionally substituted N-containing C₅₋₇ heterocyclyl group; (iii) C(═O)OR^{O1}, where R^{O1} is selected from H, optionally substituted C₁₋₇ alkyl, optionally substituted C₃₋₂₀ heterocyclyl and optionally substituted C₅₋₂₀ aryl; (iv) C(═O)NHNHSO₂R^{S1}, where R^{S1} is selected from H, optionally substituted C₁₋₇ alkyl, optionally substituted C₃₋₂₀ heterocyclyl and optionally substituted C₅₋₂₀ aryl; (v) OC(═O)RC8, where RC8 is selected from H, optionally substituted C₁₋₇ alkyl, optionally substituted C₃₋₂₀ heterocyclyl and optionally substituted C₅₋₂₀ aryl; (vi) OC(═O)NR^{N7}R^{N8}, where R^{N7} and R^{N8} are independently selected from H, optionally substituted C₁₋₇ alkyl, optionally substituted C₃₋₂₀ heterocyclyl and optionally substituted C₅₋₂₀ aryl or R^{N7} and R^{N8} and the nitrogen atom to which they are attached form an optionally substituted N-containing C₅₋₇ heterocyclyl group; and (vii) C(═O)CH₂NH C(═O)NHNH₂, CHC(CN)₂, CHC(CN)C(═O)NH₂, and carboxy; R_{C5} is selected from H, OH and NH₂; or R^{C4} and R^{C5} together with the carbon atoms to which they are bound form an optionally substituted aromatic ring containing either 5 or 6 ring atoms, of formula: where Q represents O, N, or CR^{Q1}═CR^{Q2}, where R^{Q1} and R^{Q2} are independently selected from H, OH and NH₂; R^{C6} is selected from H, OH and NH₂; and R^{C7} is selected from optionally substituted C3_12 N-containing heterocyclyl, NHC(═O)R^{C9}, CH₂NR^{N2}R^{N3} and NHC(═S)NHR^{N4}, where R^{C9} is selected from optionally substituted C₁₋₇ alkyl, optionally substituted C₃₋₂₀ heterocyclyl and optionally substituted C₅₋₂₀ aryl, R^{N2} and R^{N3} are independently selected from H, optionally substituted C₁₋₇ alkyl, optionally substituted C₃₋₂₀ heterocyclyl and optionally substituted C₅₋₂₀ aryl or R^{N2} and R^{N3} and the nitrogen atom to which they are attached form an optionally substituted N-containing C₅₋₇ heterocyclyl group, and R^{N4} is selected from optionally substituted C₁₋₇ alkyl, optionally substituted C₃₋₂₀ heterocyclyl and optionally substituted C₅₋₂₀ aryl, and when R^{C4} and R^{C5} are not bound together, R^{C3} may additionally be selected from OR⁰², where R^{O2} is a C₁₋₄ alkyl group, and C(═O)OR^{O3}, where R^{O3} is a C₁₋₄ alkyl group and R^{C2} may additionally be selected from halo, for use in stabilising a p53 protein carrying a Y220C mutation.

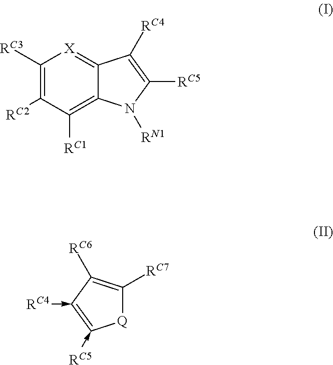

### FIELD OF THE INVENTION

The present invention relates to compounds that have the ability to bind to p53 protein molecules, and the use of such compounds.

### BACKGROUND TO THE INVENTION

The tumour suppressor protein p53 is a 393 amino acid transcription factor that regulates the cell cycle and plays a key role in the prevention of cancer development. In response to cellular stress, such as UV irradiation, hypoxia and DNA damage, p53 induces the transcription of a number of genes that are connected with G1 and G2 cell cycle arrest and apoptosis (refs 1-3). In about 50% of human cancers, p53 is inactivated as result of a mis-sense mutation in the p53 gene (refs 4,5).

The multi-functionality of p53 is reflected in the complexity of its structure. Each chain in the p53 tetramer is composed of several domains. There are well-defined DNA-binding and tetramerization domains and highly mobile, largely unstructured regions (refs 6-11). Most p53 cancer mutations are located in the DNA-binding core domain of the protein (ref 4). This domain has been structurally characterized in complex with its cognate DNA by X-ray crystallography (ref 6) and in its free form in solution by NMR (ref 12). It consists of a central β-sandwich of two anti-parallel β-sheets that serves as basic scaffold for the DNA-binding surface. The DNA-binding surface is composed of two β-turn loops (L2 and L3) that are stabilized by a zinc ion and a loop-sheet-helix motif. Together, these structural elements form an extended DNA-binding surface that is rich in positively charged amino acids and makes specific contacts with the various p53 response elements. The six amino acid residues that are most frequently mutated in human cancer are located in or close to the DNA-binding surface (cf. release R10 of the p53 mutation database at www-p53.iarc.fr)(ref 4). These residues have been classified as ‘contact’ (Arg248, Arg273) or ‘structural’ (Arg175, GIy245, Arg249, Arg282) residues, depending on whether they directly contact DNA or play a role in maintaining the structural integrity of the DNA-binding surface (ref 6).

Cancer-associated mutations are not, however, restricted to the DNA-binding surface but are also found in the β-sandwich region of the protein. The most common mutation outside the DNA-binding surface is Y220C. It is located at the far end of the β-sandwich at the start of the turn connecting β-strands S7 and S8. The benzene moiety of Tyr220 forms part of the hydrophobic core of the β-sandwich, whereas the hydroxyl group is pointing toward the solvent.

There is growing evidence that p53, which is only marginally stable at body temperature, has evolved to be highly dynamic and intrinsically unstable (ref 12).

A functional thermostable synthetic variant of p53, referred to as “T-p53C” has been used. This variant has the substitutions M133L, V203A, N239Y and N268D. This variant, which otherwise has essentially wild-type characteristics, has been used as a vector to carry various known oncogenic mutations of p53 in a form that allows determination of their structure by X-ray crystallography.

The structure of T-p53C with the Y220C mutation has been described (ref 13). With tyrosine at position 220, the benzene moiety of Tyr-220 forms part of the hydrophobic core of the beta-sandwich, whereas the hydroxyl group points toward the solvent. In the mutant when cysteine is present, the mutation creates a solvent-accessible cleft that is filled with water molecules at defined positions but leaves the overall structure of the core domain intact. The structural changes upon mutation link two rather shallow surface clefts, pre-existing in the wild type, to form a long, extended crevice in T-p53C-Y220C, which has its deepest point at the mutation site. The positions of neighbouring hydrophobic side chains located in the core of the beta-sandwich have not shifted significantly. The mutation, however, results in a loss of hydrophobic interactions and a suboptimal packing of these hydrophobic core residues. Throughout the structure however, there is no Ca displacement greater than 0.9 Å.

### DISCLOSURE OF THE INVENTION

The present inventors have discovered that a number of compounds which contain an indole, carbazole or related scaffold bind to T-p53C-Y220C and stabilizes the protein so as to increase its melting temperature.

Thus in one aspect, the invention provides a method for stabilizing a p53 protein which carries a Y220C mutation, the method comprising bringing the p53 into contact with a compound of formula (I):

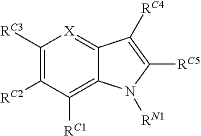

(and isomers, salts, solvates, protected forms, and prodrugs thereof) wherein X is selected from CR^{x} and N;

R^{N1} is selected from H and C₁₋₄ alkyl, which may be substituted by SH or halo;

R^{C1} is selected from H and SH;

R^{C2} is selected from H and optionally substituted C₁₋₇ alkyl;

R^{C3} is selected from H and optionally substituted C₁₋₇ alkyl;

R^{X} is selected from H, OH and NH₂;

R^{C4} is selected from:
(i) an optionally substituted C₃₋₁₂ N-containing heterocyclyl;
(ii) C(═O)NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl or R^{N5} and R^{N6} and the nitrogen atom to which they are attached form an optionally substituted N-containing C₅₋₇ heterocyclyl group;
(iii) C(═O)OR^{O1}, where R^{O1} is selected from H, optionally substituted C₁₋₇ alkyl, optionally substituted C₃₋₂₀ heterocyclyl and optionally substituted C₅₆₋₂₀ aryl ;
(iv) C(═O)NHNHSO₂R^{S1}, where R^{S1} is selected from H, optionally substituted C₁₋₇ alkyl, optionally substituted C₃₋₂₀ heterocyclyl and optionally substituted C₅₋₂₀ aryl;
(v) OC(═O)R^{C8}, where R^{C8} is selected from H, optionally substituted C₁₋₇ alkyl, optionally substituted C₃₋₂₀ heterocyclyl and optionally substituted C₅₋₂₀ aryl;
(vi) OC(═O)NR^{N7}R^{N8}, where R^{N7} and R^{N8} are independently selected from H, optionally substituted C₁₋₇ alkyl, optionally substituted C₃₋₂₀ heterocyclyl and optionally substituted C₅₋₂₀ aryl or R^{N7} and R^{N8} and the nitrogen atom to which they are attached form an optionally substituted N-containing C₅₋₇ heterocyclyl group; and
(vii) C(═O)CH₂NH₂, C(═O)NHNH₂, CHC(CN)₂, CHC(CN)C(=O)NH₂, and carboxy;

R^{C5} is selected from H, OH and NH₂;

or R^{C4} and R^{C5} together with the carbon atoms to which they are bound form an optionally substituted aromatic ring containing either 5 or 6 ring atoms, of formula:

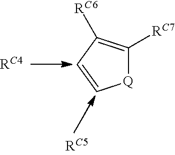

where Q represents O, N, or CR^{Q1}═CR^{Q2}, where R^{Q1} and R^{Q2} are independently selected from H, OH and NH₂;

R^{C6} is selected from H, OH and NH₂; and

R^{C7} is selected from optionally substituted C₃₋₁₂ N-containing heterocyclyl, NHC(═O)R^{C9}, CH₂NR^{N2}R^{N3} K and NHC(═S)NHR^{N4}, where R^{C9} is selected from optionally substituted C₁₋₇ alkyl, optionally substituted C₃₋₂₀ heterocyclyl and optionally substituted C₅₋₂₀ aryl, R^{N2} and R^{N3} are independently selected from H, optionally substituted C₁₋₇ alkyl, optionally substituted C₃₋₂₀ heterocyclyl and optionally substituted C₅₋₂₀ aryl or R^{N2} and R^{N3} and the nitrogen atom to which they are attached form an optionally substituted N-containing C₅₋₇ heterocyclyl group, and R^{N4} is selected from optionally substituted C₁₋₇ alkyl, optionally substituted C₃₋₂₀ heterocyclyl and optionally substituted C₅₋₂₀ aryl,

and when R^{C4} and R^{C5} are not bound together, R^{C3} may additionally be selected from OR^{O2}, where R^{O2} is a C₁₋₄ alkyl group, and C(═O)OR^{O3}, where R^{O3} is a C₁₋₄ alkyl group and R^{C2} may additionally be selected from halo.

In another aspect, the invention provides a method for treating a cell in which p53 carries a Y220C mutation, the method comprising contacting the cell with a compound of formula (I).

The above aspects may be carried out in vivo or in vitro.

In another aspect, the invention provides a method for treating a subject who has a lesion or a tumour in which p53 carries a Y220C mutation, the method comprising administering to the subject a compound of formula (I).

In another aspect, the invention provides a compound of formula (I) for use in a method of treatment of a subject who has a lesion or a tumour in which p53 carries a Y220C mutation.

The invention further provides a method of determining the binding of a molecule to a p53 which carries a Y220C mutation, the method comprising bringing the molecule into contact with said p53 in competition with a compound of formula (I), and measuring the binding or displacement of one or other of said compounds. In this aspect of the invention, one or both of the compounds may carry a label, such as a radiolabel, chromophore, fluorophore or a fluorine function for competition-based ¹⁹F-screening using magnetic resonance techniques.

Another aspect of the invention provides a pharmaceutical composition comprising a compound of formula (I) together with a pharmaceutically acceptable carrier.

Another aspect of the invention provides a compound of formula (I) for use in a method of therapy.

A further aspect of the present invention relates to novel compounds within formula (I).

### BRIEF DESCRIPTION OF THE DRAWINGS

FIG. 1 shows various representations of the crystal structure of T-p53C-Y220C in complex with a compound of the invention, PK083.

### DETAILED DESCRIPTION OF THE INVENTION

Definitions Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”) and C₁₋₇ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (0₇), octyl (C₈), nonyl (C₉), decyl (C₁₀), undecyl (C₁₁) and dodecyl (C₁₂).

Examples of (unsubstituted) saturated linear alkyl groups include, but are not limited to, methyl (CA ethyl (C₂), n-propyl (C₃), n-butyl (C₄), n-pentyl (amyl) (Cₛ), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of groups of alkenyl groups include C₂₋₄ alkenyl, C₂₋₇ alkenyl and C₂-₁₂ alkenyl.

Examples of (unsubstituted) unsaturated alkenyl groups include, but are not limited to, ethenyl (vinyl, —CH≡CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of groups of alkynyl groups include C₂₋₄ alkynyl, C₂₋₇ alkynyl and C₂₋₁₂ alkynyl.

Examples of (unsubstituted) unsaturated alkynyl groups include, but are not limited to, ethynyl (ethinyl, —C≡CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 7 carbon atoms (unless otherwise specified), including from 3 to 7 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₇ cycloalkyl and C₃₋₁₂ cycloalkyl.

Examples of cycloalkyl groups include, but are not limited to, those derived from:
saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), di methylcyclopentane (C₇), methylcyclohexane (C₇), dimethylcyclohexane (C₈) and menthane (C₁₀);
unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇) and dimethylcyclohexene (C₈);
saturated polycyclic hydrocarbon compounds:

thujane (C₁₀), carane (C₁₀), pinane (C₁₀), bornane (C₁₀), norcarane (C₇), norpinane (C₇), norbornane (C₇), adamantane (C₁₀) and decalin (decahydronaphthalene) (C₁₀); and
unsaturated polycyclic hydrocarbon compounds:

camphene (C₁₀), limonene (C₁₀) and pinene (C₁₀).

Heterocyclyl: The term “heterocyclyl”, as used herein, pertains to a monovalent moiety obtained by removing a hydrogen atom from a ring atom of a heterocyclic compound, which moiety has from 3 to 7 ring atoms (unless otherwise specified), of which from 1 to 4 are ring heteroatoms. Preferably, each ring has from 3 to 7 ring atoms, of which from 1 to 4 are ring heteroatoms.

In this context, the prefixes (e.g. C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

C₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

N-containing heterocyclyl: The term “N-containing heterocyclyl”, as used herein, pertains to a monovalent moiety obtained by removing a hydrogen atom from a ring atom of a heterocyclic compound which contains a nitrogen ring atom, which moiety has from 3 to 7 ring atoms (unless otherwise specified), of which from 1 to 4 are ring heteroatoms. Preferably, each ring has from 3 to 7 ring atoms, of which from 1 to 4 are ring heteroatoms. Examples are presented above.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇alkylacyl or C₁₋₇alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group (a C₁₋₇ alkyl ester). Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O )NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The cylic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃ , —NHC(═O)CH₂CH₃, and —NHC(═O)Ph. R¹ and R² may together form a cyclic structure, as in, for example, succinimidyl, maleimidyl, and phthalimidyl:

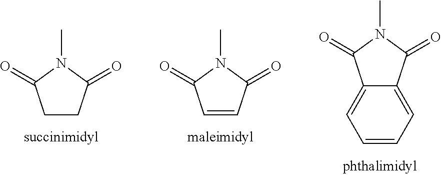

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclylgroup, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol : —SH.

Thioether (sulfide): —SR, wherein R is a thioether substituent, for example, a O₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl). The sulfone substituent may in some cases be an amino group, as defined above. These groups may be termed “aminosulfonyl” groups.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

Siloxy (silyl ether): —O SiR₃, where R is H or a C₁₋₇alkyl group. Examples of silyloxy groups include, but are not limited to, —OSiH₃, —OSiH₂(CH₃), —OSiH(CH₃)₂, —OSi(CH₃)₃ , —OSi(Et)₃, —OSi(iPr)₃, —OSi(tBu)(CH₃)₂. and —OSi(tBu)₃.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and /-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tort-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic and salt forms thereof, for example as discussed below.

Unless otherwise specified, a reference to a particular compound also includes solvates thereof, for example as discussed below.

Unless otherwise specified, a reference to a particular compound also includes prodrugs thereof, for example as discussed below.

Unless otherwise specified, a reference to a particular compound also includes protected forms thereof, for example as discussed below.

Unless otherwise specified, a reference to a particular compound also includes different polymorphic forms thereof, for example as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., “Pharmaceutically Acceptable Salts”, J. Pharm. Sci, 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO·).

For example, a carboxylic acid group may be protected as an ester for example, as: an C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₂₀ alkyl (e.g. —Me, -Et); C₁₋₇ aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O—P(═O)(OH)₂. Such a group can be cleared by phosphotase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Further Embodiments R^{N1}

In some embodiments, R^{N1} is selected from H and unsubstituted C₁₋₄ alkyl. In particular, R^{N1} may be selected from H, ethyl and propyl (e.g. iso-propyl). In other embodiments, R^{N1} may be selected from methyl and cyclopropyl

R^{C1}

In some embodiments, R^{C1} is H.

RC²

In some embodiments, R^{C2} is H.

In other embodiments, R^{C2} is optionally substituted C₁₋₇ alkyl, where the optional substituents may be selected from C₁₋₇ alkyl, C₃₋₇ heterocyclyl, C₅₋₇ aryl, halo, hydroxy, ether, nitro, cyano, acyl, carboxy, ester, amido, amino, acylamido, ureido, acyloxy and thiol.

Where R^{C2} can be halo, it may be bromo.

R^{C3}

In some embodiments, R^{C3} is H.

In other embodiments, R^{C3} is optionally substituted C₁₋₇ alkyl, where the optional substituents may be selected from C₁₋₇ alkyl, C₃₋₇ heterocyclyl, C₅₋₇ aryl, halo, hydroxy, ether, nitro, cyano, acyl, carboxy, ester, amido, amino, acylamido, ureido, acyloxy and thiol.

Where R^{C3} can be OR^{O2}, R^{O2} may be a methyl group.

In some embodiments, R^{X} is H.

R^{C4} and R^{C5}

In some embodiments, R^{C4} is an optionally substituted C₃₋₁₂ N-containing heterocyclyl and R^{C5} is selected from H, OH and NH₂. In some of these embodiments, R^{C5} is H.

In these embodiments, R^{C4} may be bound via the nitrogen ring atom or via a carbon ring atom. If R^{C4} is bound via a carbon atom, it may be monocyclic, bicyclic or tricyclic. If it is monocyclic it may be a 5- or 6-membered ring, e.g. pyrrolidine, piperidine, piperazine. A group of particular interest is based on pyrroline-2,5-dione, in which the nitrogen ring atom may be substituted, for example, by an C₅₋₆ aryl group (e.g. 4-hydroxyphenyl). If R^{C4} is tricyclic, it may be hexahydro-2,5a-diaza-cyclopenta[c]pentalen-1-one.

In other embodiments, R^{C4} is C(═O)NR^{N5}R^{N6} and R^{C5} is selected from H, OH and NH₂. In some of these embodiments, R^{C5} is H. R^{N5} and R^{N6} may be independently selected from H, optionally substituted C₁₋₇ alkyl, optionally substituted C₃₋₂₀ heterocyclyl and optionally substituted C₅₋₂₀ aryl or R^{N5} and R^{N6} and the nitrogen atom to which they are attached form an optionally substituted N-containing C₅₋₇ heterocyclyl group. In some of these embodiments, R^{N5} is selected from H and C₁₋₄ alkyl (e.g. methyl, ethyl, propyl), and R^{N6} is selected from: H, optionally substituted C₁₋₄ alkyl (e.g. methyl, ethyl, propyl, butyl), where the optional substituents may be selected from hydroxy, amino (e.g. dimethylamino) and C₅₋₉ aryl (e.g. phenyl, indolyl); and optionally substituted C₅₋₆ heterocyclyl (e.g. piperidinyl), where the optional substituents may include C₁₋₄ alkyl (e.g. methyl). In others of these embodiments, R^{N5} and R^{N6} and the nitrogen atom to which they are attached form an optionally substituted N-containing C₅₋₇ heterocyclyl group, which can be piperidinyl and piperazinyl, which may be one or more (e.g. two) optional substituents. The optional substituents may be selected from C₁₋₇ alkyl (e.g. methyl, hydroxyethyl), hydroxy, C₅₋₇ heterocyclyl (e.g. morpholino, hydroxypiperidinyl, piperidinyl, hyroxyethylpiperazinyl, piperazinyl), C₅₋₇ aryl (e.g. triazolyl, phenyl), amino (pyridylethyl-, methyl-amino) and acyl (e.g. thiophenylcarbonyl).

In other embodiments, R^{C4} is C(═O)OR^{O1} and R^{C5} is selected from H, OH and NH₂. In some of these embodiments, R^{C5} is H. R^{O1} may be optionally substituted C₁₋₇ alkyl, and preferably optionally substituted C₁₋₄ alkyl, e.g. optionally substituted methyl and ethyl. The optional substituents may be selected from ether, oxyureido (—CON(R¹)CONR²R³), C₅₋₆ aryl and amido. If the alkyl optional substituent is ether, it may be C₅₋₆ aryloxy, for example, phenoxy. The aryloxy group may itself be further substituted, for example, by an acyl (e.g. methylcarbonyl) group. If the alkyl optional substitutent is oxyureido, then the ureido substituent (R¹) may be H and the amino substituents (R², R³) may be H and a group selected from H and C₁₋₇ alkyl (e.g. methyl, ethyl, ethylenyl, cyclopentyl). If the alkyl optional substituent is C₅₋₆ aryl, it may be a C6 aryl group containing one or more nitrogen ring atoms (e.g. pyridine, pyrazine, triazine). The C₅₋₆ aryl group may itself be substituted, for example, by amino groups (e.g. NH₂, NMe₂). If the alkyl optional substituent is amdio, the amino substituents may be a C₁₋₇ alkyl group (e.g. CH₂CF₃) or a C₅₋₆ aryl group, e.g. a C₅ aryl group, such as oxazolyl.

In other embodiments, R^{C4} is C(═O)NHNHSO₂R^{S1} and R^{C5} is selected from H, OH and NH₂. In some of these embodiments, R^{C5} is H. R^{S1} may be an optionally substituted C₅₋₂₀ aryl group, and more preferably an optionally substituted C₅₋₆ aryl group, e.g. phenyl. The optional substituents may include alkoxy (e.g. OCF₃), ether (e.g. C(═O)OMe) and C₁₋₇ alkyl (e.g. CF₃).

In other embodiments, R^{C4} is OC(═O)NR^{N7}R^{N8} and R^{C5} is selected from H, OH and NH₂. In some of these embodiments, R^{C%} is H. R^{N7} may be H, and R^{N8} may be optionally substituted C₅₋₂₀ aryl, e.g. C₅₋₆ aryl (such as phenyl). The optional substituents may include halo (e.g. Cl).

In other embodiments, R^{C4} is OC(═O)R^{C8} and R^{C5} is selected from H, OH and NH₂. In some of these embodiments, R^{C5} is H. R^{C8} may be optionally substituted C₃₋₂₀ heterocyclyl (e.g. 2,3-dihydro-benzo[1,4]dioxinyl).

In other embodiments, R^{C4} is selected from C(═O)CH₂NH₂, C(═O)NHNH₂, CHC(CN)₂, CHC(CN)C(═O)NH₂, and carboxy and R^{C5} is selected from H, OH and NH₂. In some of these embodiments, R^{C5} is H.

In other embodiments, R^{C4} and R^{C5} together with the carbon atoms to which they are bound form an optionally substituted aromatic ring containing either 5 or 6 ring atoms, of formula:

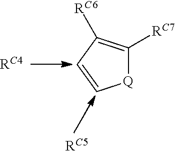

where Q represents O, N, or CR^{Q1}═CR^{C2}, where R^{Q1} and R^{Q2} are independently selected from H, OH and NH₂;

R^{C6} is selected from H, OH and NH₂; and

R^{C7} is selected from optionally substituted C₃₋₁₂ N-containing heterocyclyl, NHC(═O)R^{C9}, CH₂NR^{N2}R^{N3} and NHC(═S)NHR^{N4}.

Thus, the aromatic ring may be benzene, furan or pyrrole.

In some embodiments, R^{C6} is H.

In some embodiments, R^{C7} is an optionally substituted C₃₋₁₂ N-containing heterocyclyl. In these embodiments, R^{C7} may be bound via the nitrogen ring atom or via a carbon ring atom. If R^{C7} is bound via a carbon atom, it may be monocyclic, bicyclic or tricyclic. If it is monocyclic it may be a 5- or 6-membered ring, e.g. pyrrolidine, piperidine, piperazine. A group of particular interest is based on pyrroline-2,5-dione, in which the nitrogen ring atom may be substituted, for example, by an C₅₋₆ aryl group (e.g. 4-hydroxyphenyl). If R^{C7} is tricyclic, it may be hexahydro-2,5a-diaza-cyclopenta[c]pentalen-1-one.

In other embodiments, R^{C7} is NHC(═O)R^{C9}, where R^{C9} is selected from optionally substituted C₁₋₇ alkyl, optionally substituted C₃₋₂₀ heterocyclyl and optionally substituted C₅₋₂₀ aryl. R^{C9} may be selected from optionally substituted C₁₋₇ alkyl, and optionally substituted C₅₋₂₅ aryl.

In some of these embodiments, R^{C9} is a C₁₋₇ alkyl group, and in particular a C₁₋₄ alkyl group (e.g. methyl, ethyl, n-propyl). The optional substituents may be selected from C₁₋₇ alkyl, C₃₋₇ heterocyclyl, C₅₋₇ aryl, halo, hydroxy, ether, nitro, cyano, acyl, carboxy, ester, amido, amino, acylamido, ureido, acyloxy, thioester and thiol. In particular, the optional substituents are selected from acyloxy, C₅₋₇ aryl, amino, thioester and C₃₋₇ heterocyclyl. When the R^{C9} substituent is acyloxy, the acyloxy substituent may be selected from C₅₋₆ heterocyclyl (e.g. pyrrolidinone) and C₅₋₆ aryl (e.g. phenyl, furanyl), wherein the C₅₋₆ aryl group may bear one or more substituents (e.g. two substituents) selected from C₅₋₆aryl (e.g. tetrazolyl), acylamido (e.g. cyanomethylacylamino), nitro and ester (e.g. methylester). Further possible substituents for the C₅₋₆ aryl group include sulfonamido When the R^{C9} subsituent is acyloxy, the acyloxy substituent may also be selected from C₁₋₄ alkyl (e.g. methyl, ethylenyl), which may itself be substituted, for example by ester, acylamido or C₅₋₆ aryl. When the R^{C9} substituent is C₅₋₇ aryl, this may be a C₅ heteroaryl group (e.g. thiophenyl), which may bear, for example, and aminosulfonyl group (e.g. where the amino group is morpholino), or it may be —C₆ aryl group (e.g. pyrimidinone). When the R^{C9} substituent is amino, the amino substituents may be independently selected from C₁₋₄ alkyl (e.g. methyl, ethyl, iso-propyl). One or both of the amino susbtituents may itself be substituted, for example, by amido (e.g. —C(═O)NH₂).

Alternatively the amino group may be cyclic, for example, morpholino or piperazinyl (which may itself bear a N-susbtituent, for example amidomethyl (e.g. —CH₂—C(=O)NH₂). When the R^{C9} substituent is thioester, the ester substituent may be C₅ aryl (e.g. thiadazole) or C₆ aryl (e.g. pyrimidinone), which groups may bear an amino (e.g. —NH₂) substituent. When the R^{C9} substituent is C₃₋₇ heterocyclyl, it may be dioxo-imidzaolininyl.

In others of these embodiments, R^{C9} is a C₅₋₂₀ aryl group, and in particular a C₅₋₆ aryl group, e.g. phenyl. The optional substituents may be selected from C₁₋₇ alkyl, C₃₋₇ heterocyclyl, C₅₋₇ aryl, halo, hydroxy, ether, nitro, cyano, acyl, carboxy, ester, amido, amino, acylamido, ureido, acyloxy and thiol. In some embodiments, there is a single optional substituent, for example, ether (e.g. methoxy), which may itself be further substituted (e.g. by amido (e.g., —C(=O)NH₂)).

In others of these embodiments, R^{C9} is a C₃₋₂₀ heterocyclyl group, and in particular a C₄₋₆ heterocyclyl group, for example, 5,6-dihydro-[1,4]dioxinyl.

In other embodiments, R^{C7} is CH₂NR^{N2}R^{N3}, where R^{N2} and R^{N3} are independently selected from H, optionally substituted C₁₋₇ alkyl, optionally substituted C₃₋₂₀ heterocyclyl and optionally substituted C₅₋₂₀ aryl or R^{N2} and R^{N3} and the nitrogen atom to which they are attached form an optionally substituted N-containing C₅₋₇ heterocyclyl group.

In some embodiments, R^{N2} is selected from H and C₁₋₄ alkyl (e.g. methyl, ethyl, propyl, cyclopropyl, propenyl), and R^{N3} is selected from: optionally substituted C₁₋₄ alkyl (e.g. methyl, ethyl, propyl, butyl), where the optional substituents may be selected from hydroxy, amino (e.g. dimethylamino, ethoxyamino) and C₅₋₉ aryl (e.g. phenyl, indolyl); and optionally substituted C₅₋₆ heterocyclyl (e.g. piperidinyl), where the optional substituents may include C₁₋₄ alkyl (e.g. methyl).

In other embodiments, R^{N2} and R^{N3} and the nitrogen atom to which they are attached form an optionally substituted N-containing C₅₋₇ heterocyclyl group. In some of these embodiments, the N-containing C₅₋₇ heterocyclyl group may be piperidinyl and piperazinyl, which may bear one or more (e.g. two) optional substituents. The optional substituents may be selected from C₁₋₇ alkyl (e.g. methyl, hydroxyethyl), hydroxy, C₅₋₇ heterocyclyl (e.g. morpholino, hydroxypiperidinyl, piperidinyl, hyroxyethylpiperazinyl, piperazinyl, imidazolinonyl), C₅₋₇ aryl (e.g. triazolyl, phenyl), amino (pyridylethyl-, methyl-amino) and acyl (e.g. thiophenylcarbonyl). The optional substituents may also be selected from sulfonyl, wherein the sulfone substituent may be a C₅₋₇ aryl group (e.g. phenyl). If the optional subsitituent is a C₁₋₇ alkyl group (e.g. methyl), it may be substituted, for example, by a C₅₋₇ aryl group, such as phenyl. In others of these embodiments, the N-containing C₆₋₇ heterocyclyl group may be homopiperidinyl and homopiperazinyl, which may be substituted in a similar manner to the piperdinyl and piperazinyl groups discussed.

In further embodiments, R^{C7} is NHC(═S)NHR^{N4}, where R^{N4} is selected from optionally substituted C₁₋₇ alkyl (e.g. C₁₋₄ alkyl), optionally substituted C₃₋₂₀ heterocyclyl (e.g. C₅₋₇ heterocyclyl) and optionally substituted C₅₋₂₀ aryl (e.g. C₃₋₇ aryl). The optional substituents for these groups may be selected from C₁₋₇ alkyl, C₃₋₇ heterocyclyl, C₅₋₇ aryl, halo, hydroxy, ether, nitro, cyano, acyl, carboxy, ester, amido, amino, acylamido, ureido, acyloxy, aminosulfonyl and thiol. In some of these embodiments, R^{N4} is an optionally substituted C₅₋₆ aryl group, such as phenyl, which may bear a aminosulfonyl (e.g. dimethylaminosulfonyl) and a C₁₋₄ alkyl substituent (e.g. methyl). In others of these embodiments, R^{N4} is a C₁₋₄ alkyl group (e.g. ethyl), which may be unsubstituted.

In some embodiments, Q is CR^{Q1}═CR^{Q2}, where R^{Q1} is selected from H and OH, and R^{Q2} is H.

Certain Sets of Embodiments

In a certain set of embodiments. the compound of the invention is of formula I′:

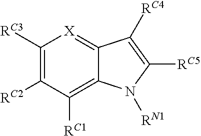

wherein X is selected from CR^{X} and N;

R^{N1} is selected from H and C₁₋₄ alkyl, which may be substituted by SH;

R^{C1} is selected from H and SH;

R^{C2} is selected from H and optionally substituted C₁₋₇ alkyl;

R^{C3} is selected from H and optionally substituted C₁₋₇ alkyl;

R^{X} is selected from H, OH and NH₂;

R^{C4} is selected from an optionally substituted C₃₋₁₂ N-containing heterocyclyl and

C(═O)NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl or R^{N5} and R^{N6} and the nitrogen atom to which they are attached form an optionally substituted N-containing C₆₋₇ heterocyclyl group;

R^{C6} is selected from H, OH and NH₂;

or R_{C4} and R^{C5} together with the carbon atoms to which they are bound form an optionally substituted aromatic ring containing either 5 or 6 ring atoms, of formula:

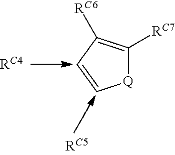

where Q represents O, N, or CR^{Q1}═CR^{Q2}, where R^{Q1} and R^{Q2} are independently selected from H, OH and NH₂;

R^{C5} is selected from H, OH and NH₂; and

R^{C7} is selected from optionally substituted C₃₋₁₂ N-containing heterocyclyl, NHC(═O)R^{C9}, CH₂NR^{N2}R^{N3} and NHC(═S)NHR^{N4}, where R^{C5} is selected from optionally substituted C₁₋₇ alkyl, optionally substituted C₃₋₂₀ heterocyclyl and optionally substituted C₅₋₂₀ aryl, R^{N2} and R^{N3} are independently selected from H, optionally substituted C₁₋₇ alkyl, optionally substituted C₃₋₂₀ heterocyclyl and optionally substituted C₅₋₂₀ aryl or R^{N2} and R^{N3} and the nitrogen atom to which they are attached form an optionally substituted N-containing C₅₋₇ heterocyclyl group, and R^{N4} is selected from optionally substituted C₁₋₇ alkyl, optionally substituted C₃₋₂₀ heterocyclyl and optionally substituted C₅₋₂₀ aryl.

In a certain set of embodiments, the compound of the invention is of formula (Ia):

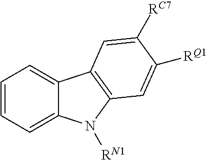

(and isomers, salts, solvates, protected forms, and prodrugs thereof) wherein R^{N1} is selected from H and C₁₋₄ alkyl;

R^{Q1} is selected from H and OH; and

R^{C7} is selected from NHC(═O)R^{C9}, CH₂NR^{N2}R^{N3} and NHC(═S)NHR^{N4}, where R^{C9} is selected from optionally substituted C₁₋₇ alkyl, optionally substituted C₃₋₂₀ heterocyclyl and optionally substituted C₅₋₂₀ aryl, R^{N2} and R^{N3} are independently selected from H, optionally substituted C₁₋₇ alkyl, optionally substituted C₃₋₂₀ heterocyclyl and optionally substituted C₅₋₂₀ aryl or R^{N2} and R^{N3} and the nitrogen atom to which they are attached form an optionally substituted N-containing C₅₋₇ heterocyclyl group, and R^{N4} is selected from optionally substituted C₁₋₇ alkyl, optionally substituted C₃₋₂₀ heterocyclyl and optionally substituted C₅₋₂₀ aryl.

In another certain set of embodiments, the compound of the invention is of formula (Ib):

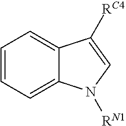

(and isomers, salts, solvates, protected forms, and prodrugs thereof)

wherein R^{N1} is selected from H and C₁₋₄ alkyl; and

R^{C4} is an optionally substituted C₃₋₁₂ N-containing heterocyclyl.

In another certain set of embodiments, the compound of the invention is of formula (Ic):

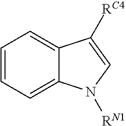

(and isomers, salts, solvates, protected forms, and prodrugs thereof)

wherein R^{N1} is selected from H and C₁₋₄ alkyl; and

R^{C4} is selecte from:
(i) an optionally substituted C₃₋₁₂ N-containing heterocyclyl;

(ii) C(═O)NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl or R^{N5} and R^{N6} and the nitrogen atom to which they are attached form an optionally substituted N-containing C₅₋₇ heterocyclyl group;
(iii) C(═O)OR^{O1, where R}^{O1} is selected from H, optionally substituted C₁₋₇ alkyl, optionally substituted C₃₋₂₀ heterocyclyl and optionally substituted C₅₋₂₀ aryl ;
(iv) C(═O)NHNHSO₂R^{S1}, where R^{S1} is selected from H, optionally substituted C₁₋₇ alkyl, optionally substituted C₃₋₂₀ heterocyclyl and optionally substituted C₅₋₂₀ aryl;
(v) OC(═O)R^{C8}, where R^{C8} is selected from H, optionally substituted C₁₋₇ alkyl, optionally substituted C₃₋₂₀ heterocyclyl and optionally substituted C₅₋₂₀ aryl;
(vi) OC(═O)NR^{N7}R^{N8}, where R^{N7} and R^{N8} are independently selected from H, optionally substituted C₁₋₇ alkyl, optionally substituted C₃₋₂₀ heterocyclyl and optionally substituted C₅₋₂₀ aryl or R^{N7} and R^{N8} and the nitrogen atom to which they are attached form an optionally substituted N-containing C₅₋₇ heterocyclyl group; and
(vii) C(═O)CH₂NH₂, C(═O)NHNH₂, CHC(CN)₂, CHC(CN)C(═O)NH₂, and carboxy;

The embodiments expressed above for R^{C4}, R^{C7} and R^{Q1} expressed above. apply to the above compounds as well:

The compounds of the examples are particular embodiments of the present invention.

Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (Eta), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

Synthesis

The compounds of the present invention are commercially available or can be readily synthesised.

Methods of the Invention

p53 Protein In the present invention, a p53 protein which carries a Y220C mutation may be the wild-type mammalian, particularly human, protein, or a stabilized version thereof. SEQ ID NO:1(AAC12971) provides the wild-type human sequence of p53. The use of human p53 is preferred.

The p53 protein may be a truncated p53 comprising the DNA-binding domain. Such a domain will generally comprise the region corresponding to residues 95 to 289 of the human sequence.

Examples of such domains are found in Joerger et al (ref 13), e.g. the region corresponding to residues 94-312 of the human sequence or a truncation thereof, such as 94-293.

Generally, where methods of the invention relate to methods such as those where p53 is provided in in vitro or other model systems, the invention may use full length or truncated p53 proteins as described above, and may incorporate one or more stabilizing alteration, e.g. one or more of the substitutions found in T-p53C. In relation to methods in of the invention relating to the treatment of lesions or tumours, the p53 will be native to the cell in which it is present. Generally, a p53 native to the cell in which it is present will correspond to the wild type sequence of p53 apart from the substitution at the position equivalent to residue 220 of SEQ ID NO:1. However, it is also possible that the protein may comprise one or more other mutations.

Methods for Stabilizing p53

In one aspect, the invention provides a method for stabilizing a p53 protein which carries a Y220C mutation, the method comprising bringing the p53 into contact with a compound of formula (I). Such a method may be practiced in vitro, e.g. by analytical centrifugation or differential scanning calorimetry, as described in the accompanying examples.

By “stabilizing p53”, it is meant increasing the melting temperature of a p53 protein having a Y220C mutation, and/or increasing the half-life of such protein.

The method of the invention may also be practiced on cells, e.g. in a cell culture of mammalian, such as human cells, wherein the cells express a p53 carrying the Y220C mutation. In the case of non-human mammalian cells, the cells may be genetically engineered to express a human p53 Y220C protein in addition to, or in place of, the native p53 protein. Cells in the culture may be primary cells, e.g. derived from a tumour of a human or non-human mammalian subject, or a cell line.

In one aspect, the above-described method may be practiced on a primary cell line or sample of a human lesion or tumour which has, or is suspected to have, a Y220C p53 protein, in order to determine the effectiveness of a compound of formula (I) in restoring or improving p53 function in the cell. For example, such an improvement or restoration may be marked by an increased rate of apoptosis in the cell culture compared to a culture of the same cells not treated with a compound of formula (I).

In a further aspect, where the method of the invention described above is practiced on a cell line or sample results in improvement or restoration of p53 function, the invention may further comprise the step of administering to the subject from whom the sample was obtained the compound of formula (I).

By “lesion” it is meant a non-cancerous growth of cells, e.g. such as a benign or pre-cancerous growth. By “tumour” it is meant any cancerous growth of a cell in which un-regulated cell division occurs at least in part as a result of the loss of p53 function caused by the presence of a Y220C mutation. In some instances, the mutation will be present together with one or more other mutations to other genes present in the cell, which will affect the growth and spread of the cancerous cells.

In some aspects the invention may be administered to a mammalian subject, such as a human, in order to treat a lesion or a tumour which has a p53 Y220C mutation. Generally, the invention will comprise administering to the subject an effective amount of a compound of formula (I) so as to improve or restore p53 function.

It is also envisaged that the invention may be practiced on a non-human animal in which a human p53 Y2200 cell line is present. This may be a xenograft cell line or the non-human animal may be a transgenic non-human mammal in which their p53 gene is replaced by a human Y220C p53 gene. Optionally, the gene may be linked to a promoter that is activatable, e.g. in a temporal fashion (i.e. at a certain point in development), in a cell-specific manner or by being induced (e.g. a tetracycline-inducible promoter).

A non-human mammal may be a rodent. Rodents include rats, mice, guinea pigs, chinchillas and other similarly-sized small rodents used in laboratory research.

The invention is not confined to any one particular type cell, but to any lesion or tumour in which p53 function is compromised by the presence of a Y220C mutation. Such a mutation may be found, for example, in leukaemias, lymphomas, myelomas, plasmacytomas, and the like; and solid tumours. Examples of solid tumours include but are not limited to colon carcinoma, pancreatic cancer, breast cancer, ovarian cancer, prostate cancer, squamous cell carcinoma, basal cell carcinoma, adenocarcinoma, renal cell carcinoma, hepatoma, cervical cancer, testicular tumour, lung carcinoma, small cell lung carcinoma, bladder carcinoma, epithelial carcinoma, melanoma, neuroblastoma, and retinoblastoma.

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g.

by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutang, gibbon), or a human.

Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, New York, USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic prdperties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate. Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

Compounds of formula (I) may be administered in conjunction with other anti-cancer agents.

Administration may be simultaneous, separate or sequential. By “simultaneous ” administration, it is meant that the compound of formula (I) and a second anti-cancer agent are administered to a subject in a single dose by the same route of administration.

By “separate” administration, it is meant that the compound of formula (I) and a second anti-cancer agent are administered to a subject by two different routes of administration which occur at the same time. This may occur for example where one agent is administered by infusion and the other is given orally during the course of the infusion.

By “sequential” it is meant that the two agents are administered at different points in time, provided that the activity of the first administered agent is present and ongoing in the subject at the time the second agent is administered. For example, another anti-cancer agent may be administered first, such that tumour cells in the subject are damaged, followed by administration of the compound of formula (I) such that p53 function is provided to induce apoptosis. Generally, a sequential dose will occur such that the second of the two agents is administered within 48 hours, preferably within 24 hours, such as within 12, 6, 4, 2 or 1 hour(s) of the first agent.

The amount of the compound of formula (I) to be administered to a subject will ultimately depend upon the nature of the subject and the disease to be treated.

A second agent may be any known agent with desirable properties having regard to the disease to be treated. Such agents include taxoids such as Taxol®, Taxotere® or other chemotherapeutics, such as cis-platin (and other platin intercalating compounds), etoposide and etoposide phosphate, bleomycin, mitomycin C, CCNU, doxorubicin, daunorubicin, idarubicin, ifosfamide, and the like. The agent may also be a biological agent such as a protein that inhibits tumour growth, such as but not limited to interferon (IFN)-gamma, tumour necrosis factor (TNF)-alpha, TNF-beta, and similar cytokines, or an anti-angiogenic factor such as angiostatin and endostatin or inhibitors of FGF or VEGF such as soluble forms of receptors for angiogenic factors, including but not limited to soluble VGF/VEGF receptor.

The invention is illustrated by the following examples.

### EXAMPLES

Experimental Procedures

Protein Expression and Purification

For crystallographic experiments, the DNA coding for residues 94-312 of T-p53C, the human p53 core domain mutant M133LN203A/N239Y/N268D was subcloned from a pRSET(A) vector into the polylinker region of a pET-24a(+) vector (Novagen) using the Ndel and EcoRl restriction sites (13). The additional point mutation of Y220C was introduced using the QuikChange Site-directed Mutagenesis kit (Stratagene) yielding “constructl”. The mutants were expressed in Escherichia coli BL21 (DE3) or C41 (DE3)—a derivative of BL21, selected for improved soluble expression of globular and membrane proteins (ref A1).

For all other experiments, the DNA coding for residues 94-312 of T-p53C was inserted into a modified pET24a(+) vector using BamHI and EcoRl restriction sites. The sequence encoding the amino acids 1-85 of the B. stearothermophilus dihydrolipoyl acetyltransferase domain (lipoyl domain, EC 2.1.12, (ref A2)) fused to a N-terminal 6×-His tag and C-terminal TEV protease cleavage site ENLYFQG(GS) (ref A3) was inserted between the Ndel and BamHl sites of pET24a(+) to make this modified vector. The additional point mutation for Y220C was introduced using the QuikChange Site-directed Mutagenesis kit (Stratagene) yielding “construct2”.

Purified plasmid DNA was submitted to Lark Technologies, Inc. (Essex) for sequencing. Both strands were sequenced using standard T7 promoter and T7 terminator primers. Both constructs (1 and 2) were confirmed to have the correct DNA sequence.

All vectors were heat pulse transformed into cryopreserved competent E. coli cells (BL21(DE3) or C41(DE3)). Freshly transformed BL21/C41 cells were grown for 12-16 hours at 37° C. on TYE/Kan/Glu agar plates. Afterwards, cell colonies were transferred into 10-1000 ml (dependent on the total volume of expression medium) of 2×TY media containing 50 μg/ml kanamycin (final concentration).This starter culture was incubated at 37° C. and 250 rpm for approximately 3 hours, or until the optical density at 600 nm (OD600) reached ˜0.5. The culture was then used as an inoculum for 2 L flasks containing 0.8 L 2×TY media or M9 minimal medium (for expression of 15N and/or 13C isotope labelled p53 for NMR studies) supplemented with a final concentration 50 μg/ml kanamycin (depending on antibiotic resistance). A 1:1000 dilution of starter culture was used to inoculate the expression cultures. The cultures were incubated at 37° C. and 250 rpm until an OD600 reached 0.6-0.9. The temperature was reduced to 18° C., the expression cultures were supplemented with 100 μM ZnSO₄, induced with 1 mM isopropyl β-D-thiogalactoside (IPTG) and grown at the induction temperature of 18° C. and 250 rpm for 14-18 hours. Cells were harvested by centrifugation for 30 min at 4500 rpm in a Sorvall RC 3B Plus rotor cooled to 4° C. If protein purification was not performed immediately, cell pellets were flash frozen in liquid nitrogen and stored at −20° C.

For the expression of ¹⁵N or ¹³C/¹⁵N labelled protein, M9 minimal medium was used instead of 2×TY according to the following receipe: 12.8 g Na₂HPO₄(anhydrous), 3.0 g NaH₂PO₄, 0.5 g NaCl, 2 ml M-MgSO₄, 2 ml SolutionQ, 1.0 g ¹⁵NH₄Cl, 30 ml g/l glucose solution or 30 ml g/l ¹³C-glucose solution and 10 ml vitamin mix. “Solution Q” consists of: 5 g FeCl₂×4H₂O, 184 mg CaCl₂×2H₂O, 64 mg H₃BO₃, 18 mg CoCl₂×6H₂O, 4 mg CuCl₂×2H₂O, 340 mg ZnCl₂, 605 mg Na₂MoO₄×2H₂O, 40 mg MnCl₂×4H₂O, 8 ml M-HCl in 1000 ml H₂O. “Vitamin mix” consists of: 50 mg Thiamine, 10 mg d-Biotin, 10 mg Choline chloride, 10 mg Folic acid, 10 mg Niacinamide, 10 mg D-Pantothenic acid, 10 mg Pyridoxal, 1 mg Riboflavin in 100 ml 1×M9 salt solution.

Purification chromatography was performed using a Biocad Vision system and an ÄKTA system. All buffers were filtered using a 0.22 μm filter before use.

“Construct 1” was purified using the following protocol:

Harvested cell pellets were first resuspended and homogenised in lysis buffer of 50 mM Tris-HCl, pH 7.2, 5mM DTT, 1 tablet/50 ml ,Complete' EDTA-free protease inhibitor as well as small amounts of DNase and RNase. This was kept on ice at all times. 25 ml of lysis buffer per litre of original cell culture was used. Cells were cracked using an Emulsiflex C5 high pressure homogeniser (Glen Creiton). The lysate was centrifuged for 40 min at 17,000 rpm in a Sorvall SS34 rotor cooled to 4° C. The supernatant was filtered using a 0.22 μm Stericup™ disposable vacuum filter device (Millipore). This was then loaded onto a Poros 20HQ cationic exchange column that had been pre-equilibrated with 25 mM NaPi, pH 7.5 +5 mM DTT, and eluted with a 0-1 M NaCl gradient over 20 column volumes. The pooled fractions were diluted tenfold with pre-chilled 25 mM NaPi, pH 7.5 +5 mM DTT, to less than 50 mM salt concentration. This was then loaded onto a Heparin HP column and eluted after 10 CV of washing by increasing the concentration of NaCl in two steps to 400 mM (5 CV) and 1 M (5 CV). The final purification step was done using a Superdex® 75 26/60 Prep Grade HiLoad column (Amersham) equilibrated with a buffer of 25 mM NaPi, pH 7.5, 150 mM NaCl and 5 mM DTT. Fractions were pooled and concentrated using a Centriprep centrifugal concentrating device (Ultracel YM-10) with a 10000 molecular weight cutoff in a Megafuge2R (Heraeus) benchtop centrifuge that was pre-cooled to 4° C. The purity of the protein was judged by SDS PAGE and was greater than 95% pure. Samples were flash frozen in liquid nitrogen and stored at −80° C.

“Construct 2” was purified using the following protocol:

Harvested cell pellets were first resuspended and homogenised in lysis buffer of 50 mM NaH₂PO₄/Na₂HPO₄-buffer (NaPi), pH 8.0, 300 mM NaCl, 10 mM imidazole, 5 mM TCEP, 1 tablet/50 ml ‘Complete’ EDTA-free protease inhibitor as well as small amounts of DNase and RNase. This was kept on ice at all times. 25 ml of lysis buffer per litre of original cell culture was used. Cells were cracked using an Emulsiflex C5 high pressure homogeniser (Glen Creston). The lysate was centrifuged for 40 min at 17,000 rpm in a Sorvall SS34 rotor cooled to 4° C. The supernatant was filtered using a 0.22 μm Stericup™ disposable vacuum filter device (Millipore). This was then loaded onto 4×5 ml HisTrap™ FF crude Ni-columns that had been pre-equilibrated with 50 mM NaPi, pH 8.0, 300 mM NaCl, 10 mM imidazole, 5 mM TCEP, and eluted with a 10-250 mM imidazole gradient over 6 column volumes. Dependent on the yield of the protein expression, the lysate was loaded in portions and/or the flowthrough was reloaded until most T-p53C-Y220C was recovered. The pooled fractions were digested with Tobacco Etch Virus protease (TEV) overnight at 4° C. cleaving the 6×HIS+Lipoyl part off the expressed protein by cutting at the TEV recognition site between ENLYFQ and GGS. The degree of cleavage was monitored by MALDI-TOF mass spectrometry and SDS-PAGE. After completion of cleavage, the solution was diluted tenfold with pre-chilled 25 mM NaPi, pH 7.5, 5 mM DTT to less than 30 mM salt concentration. This was then loaded onto a Heparin HP column and eluted after 10 CV of washing by increasing the concentration of NaCl in a gradient to 400 mM over 6 CV and then in two steps to 1M (5 CV) and 2M (5 CV). The purity of the protein was judged by SDS PAGE. If it was greater than 95% pure, the protein was directly dialysed against 25 mM NaPi, pH 7.2, 150 mM NaCl, 5 mM DTT for 6-8 hours and this was repeated at least one time after exchange of the dialysis buffer. The protein fractions that were less than 95% pure were subjected to gel filtration as a final purification step using a Superdex® 75 26/60 Prep Grade HiLoad column (Amersham) equilibrated with a buffer of 25 mM NaPi, pH 7.2, 150 mM NaCl and 5 mM DTT. Fractions were pooled and concentrated using a Centriprep centrifugal concentrating device (Ultracel YM-10) with a 10000 Mᵣ cutoff in a Megafuge2R (Heraeus) benchtop centrifuge that was pre-cooled to 4° C. The purity of the protein was judged by SDS PAGE and was greater than 95% pure. Samples were flash frozen in liquid nitrogen and stored at −80° C.

Protein concentrations were measured spectrophotometrically as described by Gill and von Hippel (ref A4). The molar extinction coefficient for T-p53C-Y220C at 280 nm (ε²⁸⁰) _{was} calculated to be ε²⁸⁰=16590 cm⁻¹M⁻¹ from its amino acid sequence.

Sample Preparation and NMR Screening Using ¹H/¹⁵N-HSQC

Uniformly ¹⁵N-labelled core domains of T-p53C-Y220C was expressed and purified according to the above described protocol. The low molecular-weight compounds were dissolved in d⁶-DMSO to make 10 mM stock solutions. To screen compound mixtures by chemical shift mapping, 10 μl of each of 4 different compounds were mixed together and 25 μl of this mixture was added to 25 μl of D₂O and 500 μl of 70 μM T-p53C-Y220C (in 25 mM NaPi, 150 mM NaCl and 5 mM DTT, pH 7.2 ). The final concentration for each compound was 114 μM at concentration of 4.5% (v/v) d⁶-DMSO. NMR samples were freshly prepared and kept sealed under argon after degassing by repeated cycles of pumping the NMR tube under low pressure (while gently tapping the tube) and returning to atmospheric pressure using a stream of argon gas. This was done to maintain sample stability.

¹H/¹⁵N HSQC spectra were acquired at 293K on Bruker Avancell+700 and Avance 800 spectrometers using a ¹H/¹³C/¹⁵N triple resonance inverse, cryogenic 5 mm probe (Bruker), with the following parameters: 16 scans, 128 complex points in t1, recycle time of 0.95 seconds, and 1024 total points in t2. Using Bruker's TopSpin 2.0 software, the number of complex points in t1 was doubled by forward complex linear prediction and shifted squared sine bell window functions were applied to both dimensions prior to zero filling and Fourier transformation. A digital resolution of 2.0 Hz/point in the ¹H frequency dimension and 4.7 Hz/point in the ¹⁵N frequency dimension was used. Spectra were analysed using Sparky 3.113 (A5). Chemical shifts were considered significant if the average weighted ¹H/¹⁵N chemical shift difference (Δδ(¹H/¹⁵N)=|Δδ(¹H)|+|Δδ¹⁵N|)/5) was greater than 0.04 ppm (Hajduk et al., 1997). An in-house script was used to analyse chemical shift differences and map these onto PDB protein structures.

Where binding was detected, deconvolution was accomplished by screening four separate samples of individual compounds, each present at a final concentration of 227 μM.

For certain compounds an array of different concentrations were used to derive K_{D} values (K_{D} NMR) by fitting a saturation binding equation (Δδ=c+a*[L]/(K_{D}+[L])) to the concentration-dependent chemical shift changes of the relevant shifting peaks.

Thermal Denaturation Studies Using Capillary DSC

Differential Scanning calorimetry (DSC) was used to probe the stabilising effect of the ligands on T-p53C-Y2200. For a reversible two-state system, the melting temperature, Tₘ, is the transition temperature where the folded and unfolded states are equally populated. However, p53 does not denature reversibly with increasing temperature and so the observed Tₘ is not a true melting temperature, but an apparent one (Tₘ^{aPP}) and derived data are semi-quantitative, depending upon the rate of heating.

DSC experiments were performed using a Microcal VP-Capillary DSC instrument (Microcal, Amherst, Mass.) with an active cell volume of ˜125 μl. Protein samples were buffer exchanged into 25 mM NaPi, pH 7.2, 150 mM NaCl, 5 mM DTT. This buffer + the respective concentration of ligand/DMSO was used for baseline scans, so that the only difference between sample and reference cell or sample cell in the measurement and both cells in the baseline scan is the presence of the protein. A final concentration of 20 μM T-p53C-Y220C was used. A pressure of 2.5 bars (nitrogen) was applied to the cell. Temperatures from 10 to 85° C. were scanned at a rate of 250° C./h, with a filtering period of 4 seconds and feedback gain/mode set at medium. Data were analysed with ORIGIN software (Microcal).

Thermal Denaturation Studies Using Fluorescence

This was by an adaption of the classical procedure (e.g. ref A6). Thermal unfolding was monitored by the binding of the dye Sypro Orange (5×) using a Rotor-gene 6000 (Corbett Life Science) at 270 K/h in 25 mM NaPi, 150 mM NaCl and 5 mM DTT, pH 7.2 with a protein concentration of 10 μM.

Measuring binding of small molecules to T-p53C-Y220C by analytical ultracentrifugation (AUC)

Analytical centrifugation studies the distribution of molecules under gravity force. In equilibrium sedimentation experiment the solute concentrates at the bottom of the cell and forms a concentration gradient. The steepness of this gradient depends on the molecular weight of the solute, the heavier the molecule the steeper the gradient. Small molecules have a molecular weight around 500 Da, and p53 core domain is large protein molecule of 24.5 kDa. Under the experimental conditions chosen, small molecules form a very shallow, practically negligible gradient, while p53 presents a very well defined sedimentation profile. If the small molecule is bound to p53, it displays the sedimentation profile of p53. By monitoring the distribution of the small molecule via absorbance at wavelengths above 300 nm (the ligands must absorb light in this spectral range for the method to work) so that signal is not affected by the absorbance of the protein (e.g., 310, 340 and 380 nm), it is possible to determine if the small molecule binds to the protein, and in many cases measure the dissociation constant. This method is uniquely suited for measuring weak (10 μM-1 mM) dissociation constants as it does not require complete binding of ligand by protein. It works best when concentration of protein is approximate equal to the dissociation constant. For detailed description of the data analysis see ref.(ref A7).

Equilibrium sedimentation experiments were performed on a Beckman XL-I ultracentrifuge using Ti-50 rotor and 6-sector cells. at speeds of 30,000 and 40,000 rpm at 10° C. Up to 21 samples were analysed simultaneously. Buffer conditions were 25 mM NaPi, 150 mM NaCl, 5 mM DTT. The samples contained ligand at concentration of 15 μM-40 μM, with absorbance of 0.3-0.5 at one of the selected wavelengths and 100 μM T-p53-Y220C.

Time-dependent Fluorescence Studies

Unfolding kinetics was performed as described by Friedler et al. (ref A8) at 37° C. in 50 mM Hepes, pH 7.2, 1 mM Tris-2-carboxyethylphosphine (TCEP), by following the emission of tryptophan at 340 nm on excitation at 280 nm, using a Cary Eclipse fluorescence spectrophotometer controlled by the supplied Cary software.

Compounds

All the compounds tested were obtained from ENAMINE Ltd. (23 Alexandra Matrosova Street, 01103 KIEV, Ukraine), except for PK 390-392 which were obtained from Asinex and PK402, 407 and 408 which were obtained from InterBioScreen.

Results

The following tables show the results of the thermal denaturation studies using capillary DSC, where ΔTₘ is the increase in Tm on adding 250 μM of the test compound. The Dunnett significance test (P) value given is the calculated probability that the change in Tₘ is insignificant.

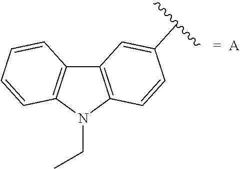

| | | | P (Dunnett |
|---|---|---|---|
| Structural formula | Lig-ID | ΔTₘ | post hoc) |
| 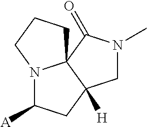 | PK226 | 1.11 | <.0001 |
| 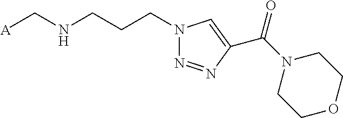 | PK219 | 0.85 | <.0001 |
| 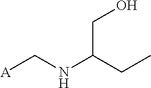 | PK209 | 0.79 | 0.0002 |
| 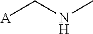 | PK083 | 0.75 | <0.0001 |
| 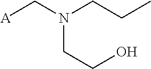 | PK191 | 0.70 | <.0001 |
| 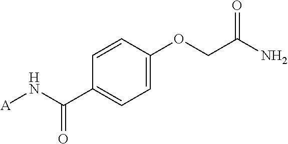 | PK166 | 0.64 | <0.0001 |
| 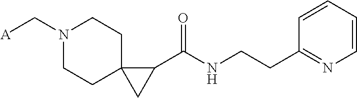 | PK220 | 0.64 | <.0001 |
| 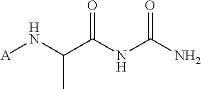 | PK158 | 0.63 | <0.0001 |
| 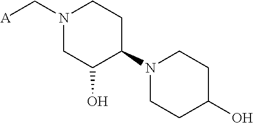 | PK227 | 0.60 | <.0001 |
| 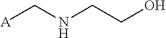 | PK211 | 0.58 | 0.0087 |
| 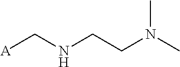 | PK207 | 0.57 | 0.0097 |
| 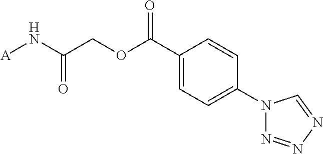 | PK138 | 0.54 | <0.0001 |
| 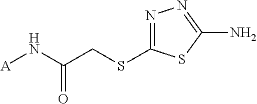 | PK099 | 0.53 | <0.0001 |
| 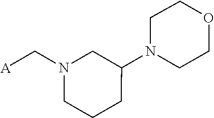 | PK225 | 0.53 | <.0001 |
| 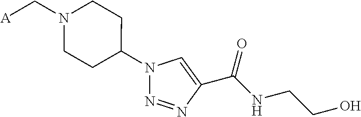 | PK221 | 0.52 | <.0001 |
| 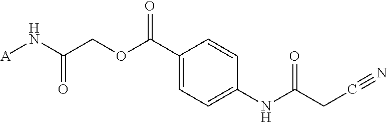 | PK140 | 0.51 | <0.0001 |
| 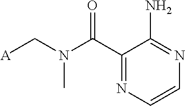 | PK164 | 0.51 | <0.0001 |
| 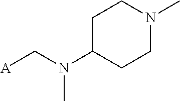 | PK178 | 0.50 | 0.0004 |
| 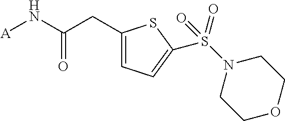 | PK115 | 0.49 | 0.0002 |
| 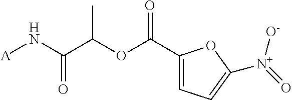 | PK119 | 0.48 | 0.0002 |
| 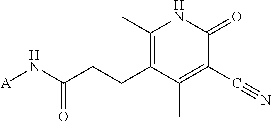 | PK141 | 0.48 | <0.0001 |
| 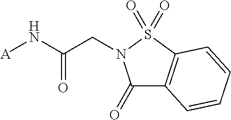 | PK117 | 0.47 | 0.0003 |
| 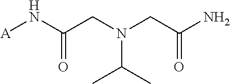 | PK161 | 0.47 | 0.0003 |
| 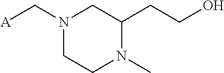 | PK217 | 0.47 | <.0001 |
| 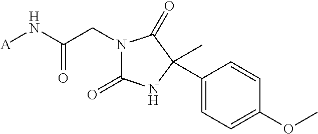 | PK160 | 0.42 | 0.0028 |
| 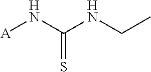 | PK168 | 0.41 | 0.0017 |
| 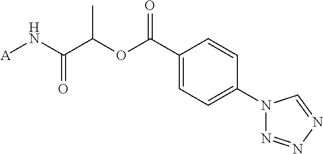 | PK134 | 0.40 | <0.0001 |
| 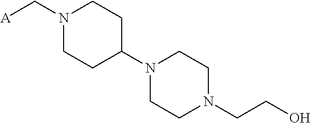 | PK205 | 0.40 | 0.1023 |
| 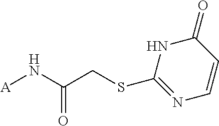 | PK095 | 0.39 | 0.0059 |
| 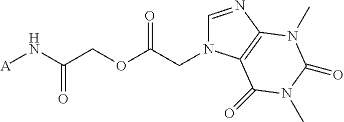 | PK114 | 0.39 | 0.0045 |
| 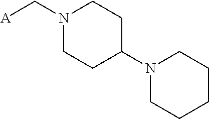 | PK188 | 0.39 | <.0001 |
| 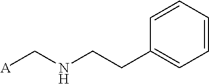 | PK194 | 0.38 | <.0001 |
| 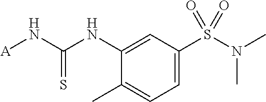 | PK102 | 0.37 | 0.0082 |
| 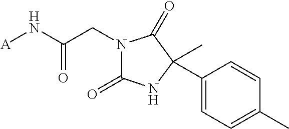 | PK110 | 0.37 | 0.0071 |
| 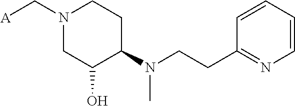 | PK216 | 0.34 | 0.0001 |
| 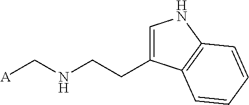 | PK193 | 0.30 | <.0001 |
| 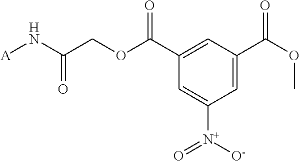 | PK137 | 0.29 | 0.0013 |
| 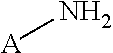 | PK150 | 0.27 | 0.0002 |
| 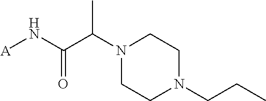 | PK142 | 0.26 | 0.0001 |
| 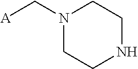 | PK198 | 0.25 | 0.0013 |
| 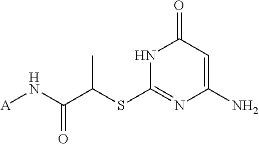 | PK143 | 0.24 | 0.0007 |
| 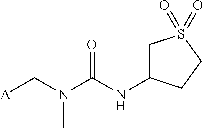 | PK148 | 0.23 | 0.0013 |
| 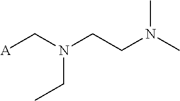 | PK189 | 0.23 | 0.003 |
| 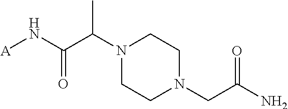 | PK152 | 0.22 | 0.0023 |

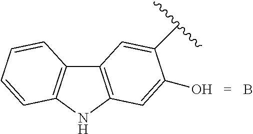

| | | | P |
|---|---|---|---|
| | | | (Dunnett |
| Structural formula | Lig-ID | ΔTₘ | post hoc) |
| 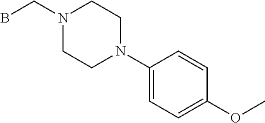 | PK144 | 0.51 | <0.0001 |
| 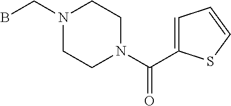 | PK145 | 0.36 | <0.0001 |
| 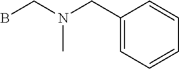 | PK147 | 0.34 | <0.0001 |

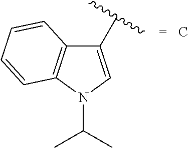

| | | | P |
|---|---|---|---|
| | | | (Dunnett |
| Structural formula | Lig-ID | ΔTₘ | post hoc) |
| 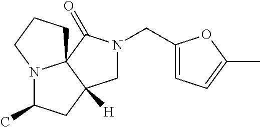 | PK223 | 0.38 | 0.0002 |

¹H/¹⁵N-HSQC

Compound PK059:

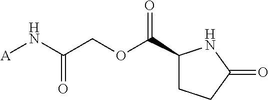

| Compound | K_{D} (@ 20° C.) |
|---|---|
| PK059 | 213 μM |
| PK083 | 167 μM |

AUC

| Compound | K_{D} (@ 10° C.) |
|---|---|
| PK059 | 300 μM |
| PK083 | 170 μM |

Thermal Stabilization and Kinetics of Denaturation

It was observed from differential scanning calorimetry that PK083 stabilized T-p53C-Y220C in a concentration-dependent manner. T-p53C-Y220C denatures irreversibly and its apparent Tₘ varies with heating rate. At very fast heating, the measured Tₘ approximates to its true value, since the irreversible process is slower than equilibration. The Tₘ is raised nearly 2° C. from 316 K by 2.5 mM PK083, and the data fit the equation expected for stabilization by simple binding with an approximate K_{D} of 140±73 μM at 316-318 K.

The kinetics of denaturation of T-p53C-Y220C at 310 K (37° C.) was fitted to a simple binding model for PK083. In the absence of ligand, the protein had a half-life of 3.8 min. This increased to 15.7 min at saturating concentrations of PK083.

X-ray Crystallography Methods

Crystals of T-p53C-Y220C in space group P2₁2₁2₁ with two molecules in the asymmetric unit were grown at 21° C. by sitting drop vapour diffusion under the conditions described previously (ref B1). PK083 was soaked into crystals of T-p53C-Y220C by stepwise addition of cryo buffer (19% polyethylene glycol 4,000, 20% glycerol, 100 mM Hepes, pH 7.2, 150 mM KCl) with increasing concentration of PhiKan083 over a period of 2 hours. After reaching the final concentration of 10 mM, soaking was continued for another 30 minutes before the crystals were flash frozen in liquid nitrogen. An X-ray data set to 1.5-A resolution was collected at 100 K on beamline 104 at the Diamond Light Source. Data processing was performed using Mosflm (ref B2) and Scala (ref B3). Structure solution and refinement were performed with CNS (ref B4). After an initial round of rigid body refinement using the structure of free T-p53C-Y220C (PDB entry 2J1X) as starting model, the structure of the complex was refined by iterative cycles of refinement with CNS and manual model building with MAIN (ref B5). Water molecules were added to the structure using the water pick option implemented within CNS and manual model building. At this stage of the refinement, PK083 was built into the model of chain B, and the structure was further refined, including incorporation of alternative conformations for selected side chains. For the cavity in chain A, significant difference density was observed having contributions from PK083 in the same binding mode as in chain B but bound with a low occupancy and a network of water molecules in the unbound state (coordinates not included in the final model). The data collection and refinement statistics are shown in Table 1.

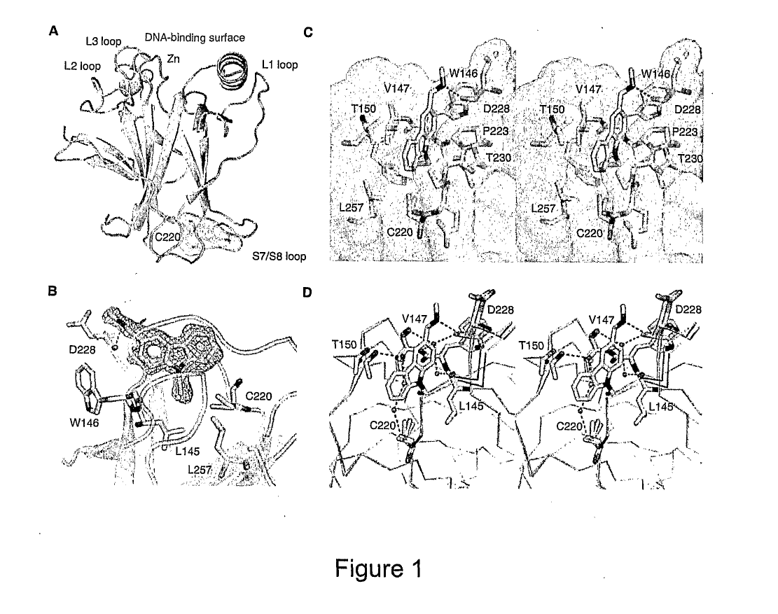

The crystal structure of T-p53C-Y220C in complex with PK083 is shown in FIG. 1. FIG. 1A is a ribbon representation of the overall structure of T-p53C-Y220C (chain B) in complex with PK083. PK083 is shown in green as a stick model with its molecular surface. It binds to the mutation-induced cleft on the protein surface that is distant froM the known functional interfaces of the protein. The side chain of Cys220 at the mutation site, which adopts two alternative conformations, is highlighted in orange. FIG. 1B is a |Fₒ-F_{c}| simulated annealing omit map of PK083 bound to chain B of T-p53C-Y220C contoured at 3.0 σ. FIG. 1C is a stereo view of the PhiKan083 binding site. Selected p53 residues within a 5-Å distance of the ligand are shown as grey stick models. The protein surface is highlighted in semitransparent grey. FIG. 1D is a superposition of T-p53C-Y220C in its free (PDB code 2J1X chain B; green) and PK083-bound form (yellow), indicating small structural shifts upon ligand binding. PK083 is depicted as a grey stick model. The small red spheres indicate water molecule in the ligand-free structure that are displaced upon ligand binding.

The central carbazole moiety is largely buried in the cleft, with the 9-ethyl group occupying the deepest part of the hydrophobic pocket (FIG. 1C). Binding would appear to have an important contribution from hydrophobic packing interactions. The ethyl group is in close contact to the sulfhydryl group of the mutated residue Cys220, which adopts two alternative conformations, and a number of hydrophobic side chains (Phe109, Leu145, Va1147, and Leu257), thus anchoring the ligand to the pocket. The planar carbazole ring system is sandwiched between the hydrophobic side-chains of Pro222 and Pro223 on one side, and Va1147 and Pro151 on the other side of the binding cleft. The ring nitrogen sits close to the position of the hydroxyl group of the tyrosine residue in the wild-type structure (1.0-Å distance). The N-methylmethanamine moiety forms a hydrogen bond with the main-chain carbonyl of Asp228 (2.8-A distance). Only very small structural shifts occur upon ligand binding to the mutant. The residues that are within 5 Å of PhiKan083 (residues 109, 145-147, 150, 151, 220-223, 228-230, and 257) superimpose with a root mean square deviation of 0.3 Å (all atoms). The most significant shift is observed for the side chain of Thr150, which is displaced by up to 1.4 Å upon binding, thus widening the entrance of the pocket (FIG. 1D).

TABLE 1
Data collection and refinement statistics

| | T-p53C-Y220C-PK083 |
|---|---|
| A. Data Collection | |
| Space Group | P2₁2₁2₁ |
| Cell, a, b, c (Å) | 65.09, 71.23, 105.21 |
| Molecules per asymmetric unit | 2 |
| Resolution (Å)^{b} | 65.1 − 1.50 |
| | (1.58 − 1.50) |
| Unique reflections | 76,025 |
| Completeness (%)^{a} | 96.6 (83.4) |
| Multiplicity | 5.6 (4.6) |
| R_{merge}(%)^{a, b} | 6.6 (22.9) |
| <\|/σₗ>^{a} | 17.7 (5.6) |
| Wilson B value (Å²) | 13.8 |
| B. Refinement | |
| Number of atoms | |
| Proteins^{c} | 3119 |
| Water | 439 |
| Zinc | 2 |
| PhiKan083 | 18 |
| R_{cryst}, (%)^{d} | 18.6 |
| R_{free}, (%)^{d} | 20.8 |
| R.m.s.d. bonds (Å) | 0.009 |
| R.m.s.d. angles (°) | 1.5 |
| Mean B value (Å²) | 16.0 |
| Ramachandran plot statistics^{e} | |
| Most favored/additional allowed (%) | 91.9/8.1 |

^{a}Values in parentheses are for the highest resolution shell.
^{b}R_{merge} = Σ(I_{h, i} − <Iₕ>)/ΣI_{h, i}
^{e}Number includes alternative conformations.
^{d}R_{cryst} and R_{free} = Σ||F_{obs}| − |F_{calc}||/Σ|F_{obs}| where R_{free} was calculated over 5% of the amplitudes chosen at random and not used in the refinement.
^{e}Calculated with PROCHECK (B6).

Further Results

The indole derivative 1H-indole-3-carboxamide:

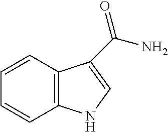

and N-(9-Ethyl-9H-carbazol-3-yl)-2,2,2-trifluoro-acetamide

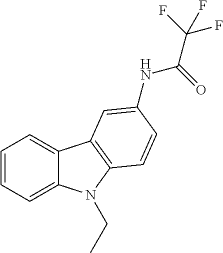

have been shown to bind to T-p53C-Y220C by NMR spectroscopy and to raise its melting temperature.

Further thermal denaturation studies using fluorescence were carried out on the following compounds described above, yielding the results as set out in the table below. Where listed, S.E. is the calculated standard error.

| Lig-ID | ΔTₘ (° C.) | S.E. (ΔTₘ) |
|---|---|---|
| PK226 | 1.0 | 0.04 |
| PK219 | 0.873 | 0.087 |
| PK209 | 0.793 | 0.02 |
| PK083 | 0.76 | 0.064 |
| PK191 | 0.7 | 0.011 |
| PK220 | 0.68 | 0.015 |
| PK166 | 0.64 | |
| PK158 | 0.63 | |
| PK227 | 0.62 | 0.046 |
| PK211 | 0.58 | 0.02 |
| PK207 | 0.57 | 0.048 |
| PK225 | 0.55 | |
| PK138 | 0.54 | |
| PK221 | 0.54 | 0.03 |
| PK099 | 0.53 | |
| PK140 | 0.513 | |
| PK164 | 0.51 | |
| PK217 | 0.493 | 0.015 |
| PK115 | 0.49 | |
| PK119 | 0.48 | |
| PK141 | 0.48 | |
| PK117 | 0.47 | |
| PK161 | 0.46 | |
| PK160 | 0.42 | |
| PK134 | 0.4 | |
| PK168 | 0.4 | |
| PK205 | 0.4 | |
| PK114 | 0.39 | |
| PK188 | 0.39 | |
| PK194 | 0.38 | |
| PK102 | 0.37 | |
| PK110 | 0.37 | |
| PK216 | 0.37 | |
| PK178 | 0.34 | 0.029 |
| PK137 | 0.3 | |
| PK193 | 0.3 | |
| PK150 | 0.27 | |
| PK142 | 0.26 | |
| PK198 | 0.25 | |
| PK143 | 0.24 | |
| PK163 | 0.24 | |
| PK189 | 0.23 | |
| PK148 | 0.225 | |
| PK152 | 0.22 | |
| PK144 | 0.51 | |
| PK145 | 0.36 | |
| PK147 | 0.34 | |
| PK223 | 0.40 | |

Thermal denaturation studies using fluorescence were carried out on the following compounds yielding the results as set out in the table below. Where listed, S.E. is the calculated standard error.

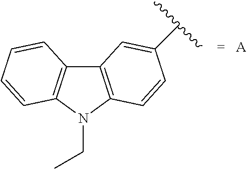

| Structural formula | Lig-ID | ΔTₘ (° C.) | S.E. (ΔTₘ) |
|---|---|---|---|
| 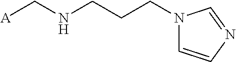 | PK214 | 0.9 | 0 |
| 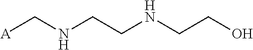 | PK392 | 0.70 | |
| 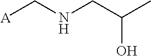 | PK391 | 0.66 | |
| 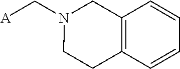 | PK176 | 0.455 | |
| 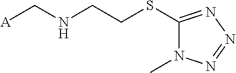 | PK390 | 0.41 | |
| 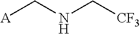 | PK230 | 0.4 | |
| 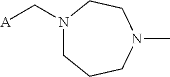 | PK175 | 0.395 | |
| 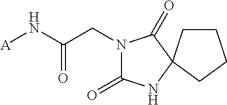 | PK116 | 0.365 | |
| 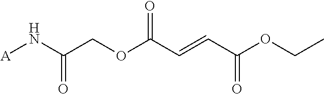 | PK113 | 0.36 | |
| 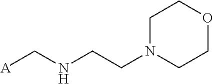 | PK208 | 0.35 | |
| 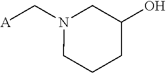 | PK180 | 0.34 | |
| 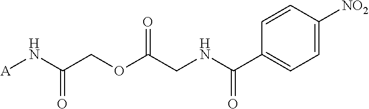 | PK108 | 0.335 | |
| 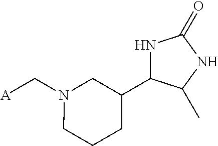 | PK218 | 0.31 | |
| 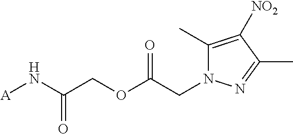 | PK118 | 0.28 | |
| 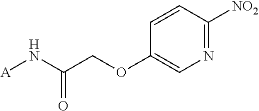 | PK163 | 0.27 | |
| 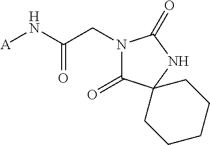 | PK165 | 0.24 | |
| 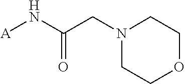 | PK128 | 0.24 | |
| 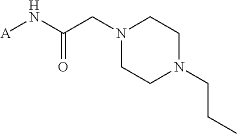 | PK126 | 0.23 | |
| 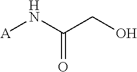 | PK132 | 0.2 | |
| 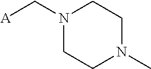 | PK177 | 0.2 | |
| 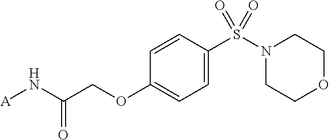 | PK094 | 0.195 | |
| 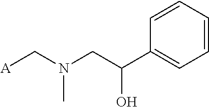 | PK186 | 0.18 | |
| 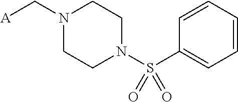 | PK215 | 0.17 | |
| 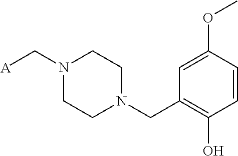 | PK202 | 0.17 | |
| 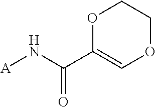 | PK153 | 0.16 | |
| 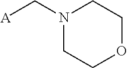 | PK190 | 0.15 | |
| 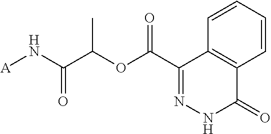 | PK133 | 0.145 | |
| 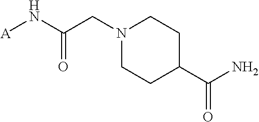 | PK151 | 0.14 | |
| 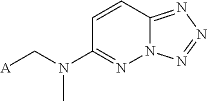 | PK157 | 0.12 | |
| 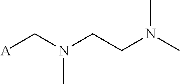 | PK183 | 0.11 | |
| 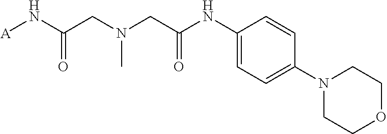 | PK122 | 0.28 | |
| 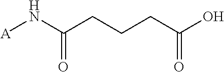 | PK135 | 0.083 | |
| 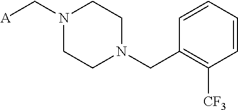 | PK197 | 0.08 | |

| 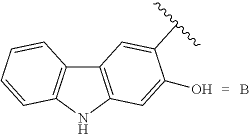 | | |
|---|---|---|
| Structural formula | Lig-ID | ΔTₘ (° C.) |
| 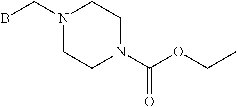 | PK149 | 0.115 |
| 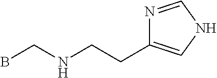 | PK146 | 0.087 |

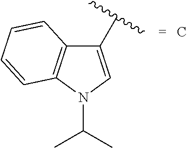

| Structural formula | Lig-ID | ΔTₘ (° C.) |
|---|---|---|
| 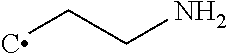 | PK235 | 0.34 |
| 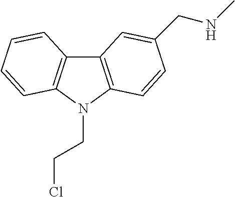 | PK229 | 0.7 |
| 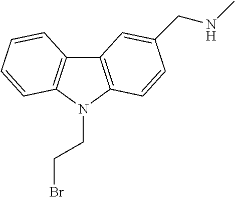 | PK232 | 0.39 |
| 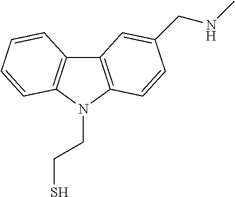 | PK234 | 0.28 |

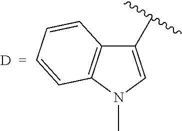

| Structural formula | Lig-ID | ΔTₘ (° C.) |
|---|---|---|
| 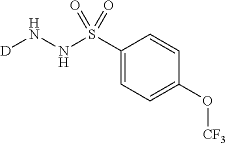 | PK254 | 0.43 |
| 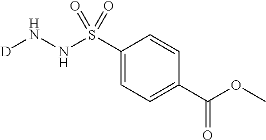 | PK281 | 0.43 |
| 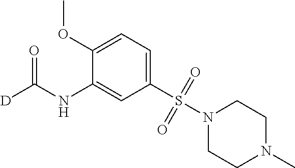 | PK280 | 0.42 |
| 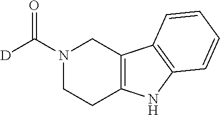 | PK293 | 0.3741 |
| 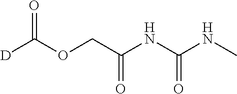 | PK268 | 0.31 |
| 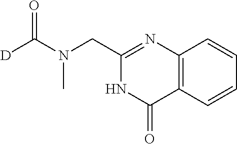 | PK315 | 0.29 |
| 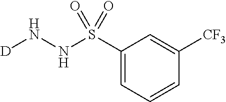 | PK255 | 0.26 |
| 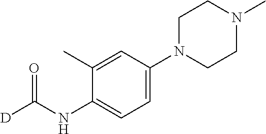 | PK295 | 0.244 |
| 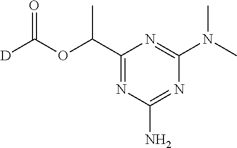 | PK286 | 0.24 |
| 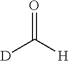 | PK249 | 0.23 |
| 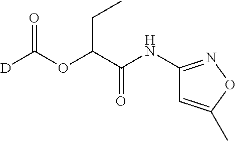 | PK284 | 0.23 |
| 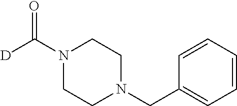 | PK285 | 0.2 |
| 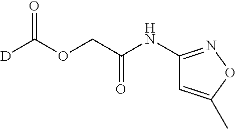 | PK264 | 0.19 |
| 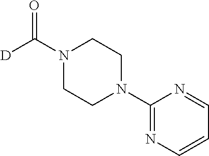 | PK283 | 0.19 |
| 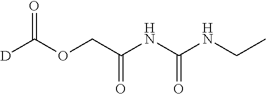 | PK266 | 0.18 |
| 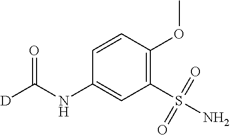 | PK292 | 0.154 |
| 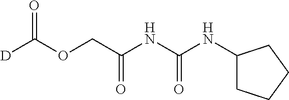 | PK269 | 0.15 |
| 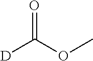 | PK316 | 0.12 |
| 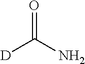 | PK320 | 0.12 |
| 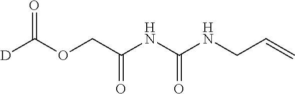 | PK256 | 0.1 |
| 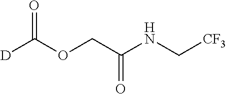 | PK258 | 0.1 |
| 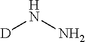 | PK253 | 0.093 |
| 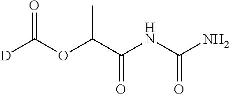 | PK263 | 0.06 |

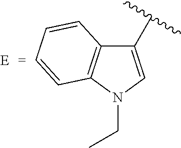

| | | ΔTₘ |
|---|---|---|
| Structural formula | Lig-ID | (° C.) |
| 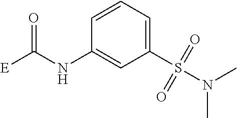 | PK272 | 0.44 |
| 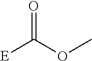 | PK339 | 0.4 |
| 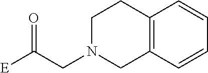 | PK212 | 0.387 |
| 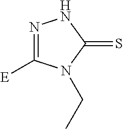 | PK236 | 0.3 |
| 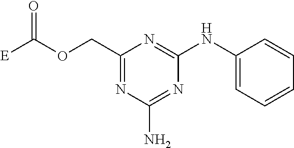 | PK301 | 0.3 |
| 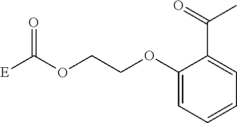 | PK309 | 0.29 |
| 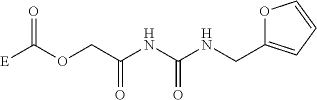 | PK273 | 0.28 |
| 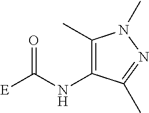 | PK313 | 0.28 |
| 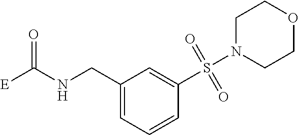 | PK306 | 0.26 |
| 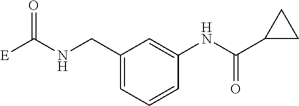 | PK307 | 0.26 |
| 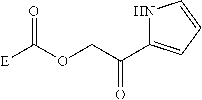 | PK279 | 0.24 |
| 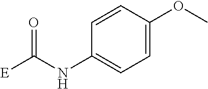 | PK237 | 0.2 |
| 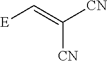 | PK238 | 0.2 |
| 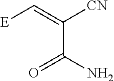 | PK240 | 0.2 |
| 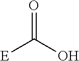 | PK242 | 0.16 |
| 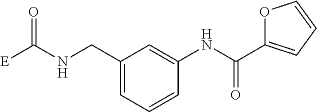 | PK314 | 0.16 |
| 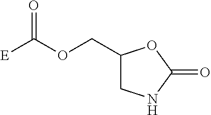 | PK311 | 0.15 |
| 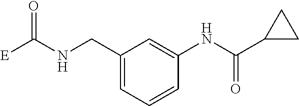 | PK312 | 0.15 |
| 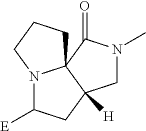 | PK222 | 0.14 |
| 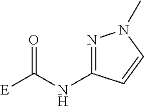 | PK318 | 0.12 |
| 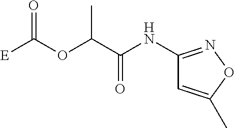 | PK270 | 0.11 |
| 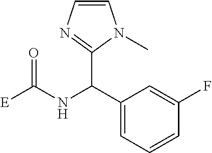 | PK317 | 0.09 |

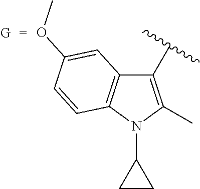

| Structural formula | Lig-ID | ΔTₘ (° C.) |
|---|---|---|
| 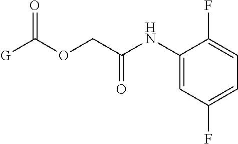 | PK310 | 0.36 |
| 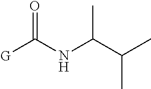 | PK277 | 0.34 |
| 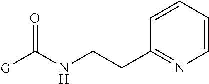 | PK276 | 0.29 |
| 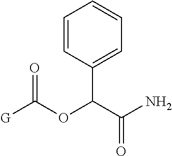 | PK294 | 0.064 |
| 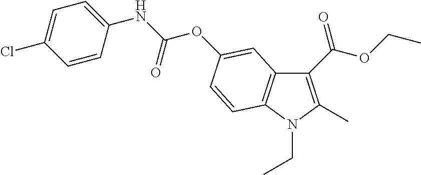 | PK246 | 0.31 |
| 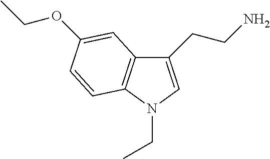 | PK407 | 0.29 |
| 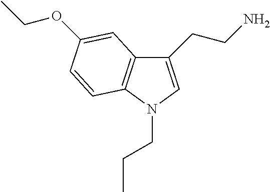 | PK408 | 0.26 |
| 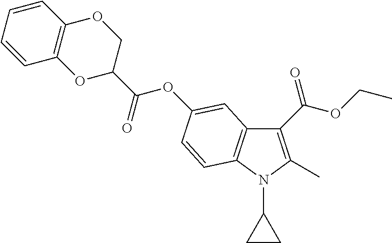 | PK305 | 0.26 |
| 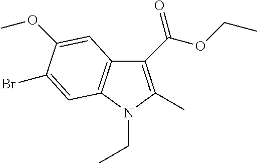 | PK248 | 0.21 |
| 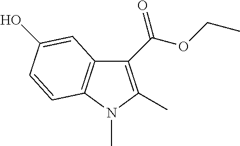 | PK241 | 0.2 |
| 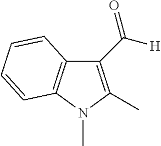 | PK243 | 0.2 |
| 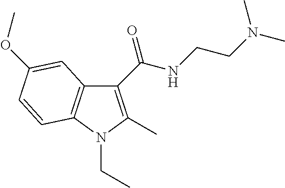 | PK402 | 0.19 |
| 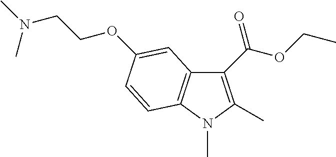 | PK250 | 0.14 |

Further studies to measure the binding of certain compounds were carried out using the NMR technique described above, with some compound being tested further to yield a revised values. The results are set out below.

| Compound | K_{D} (@ 20° C.) μM |
|---|---|
| PK226 | 113 |
| PK214 | 76 |
| PK209 | 97 |
| PK083 | 114 |
| PK211 | 120 |
| PK207 | 200 |
| PK328 | 2941 |

### REFERENCES

1. Vogelstein, B., Lane, D., and Levine, A. J. (2000) Nature 408, 307-310

2. Ryan, K. M., Phillips, A. C., and Vousden, K. H. (2001) Curr. Opin. Cell Biol. 13, 332- 337

3. Vousden, K. H., and Lu, X. (2002) Nat. Rev. Cancer 2, 594-604

4. Olivier, M., Eeles, R., Hollstein, M., Khan, M. A., Harris, C. C., and Hainaut, P. (2002) Hum Mutat 19, 607-614

5. Beroud, C., and Soussi, T. (2003) Hum Mutat 21, 176-181

6. Cho, Y., Gorina, S., Jeffrey, P. D., and Pavfetich, N. P. (1994) Science 265, 346-355

7. Clore, G. M., Ernst, J., Clubb, R., Omichinski, J. G., Kennedy, W. M., Sakaguchi, K., Appella, E., and Gronenborn, A. M. (1995) Nat Struct Biol 2, 321-333

8. Jeffrey, P. D., Gorina, S., and Pavletich, N. P. (1995) Science 267, 1498-1502

9. Bell, S., Klein, C., Muller, L., Hansen, S., and Buchner, J. (2002) J Mol Biol 322, 917-927

10. Dawson, R., Muller, L., Dehner, A., Klein, C., Kessler, H., and Buchner, J. (2003) J Mol Biol 332, 1131-1141

11. Veprintsev, D. B., Freund, S. M., Andreeva, A., Rutledge, S. E., Tidow, H., Canadillas, J. M., Blair, C. M., and Fersht, A. R. (2006) Proc Natl Acad Sci USA 103, 2115-2119

12. Canadillas, J. M., Tidow, H., Freund, S. M., Rutherford, T. J., Ang, H. C., and Fersht, A. R. (2006) Proc Natl Acad Sci U S A 103, 2109-2114

13. Joerger, A. C., Ang, H. C., and Fersht, A. R. (2006) Proc Natl Acad Sci U S A 103, 15056-15061

14. Weinberg R L, Veprintsev D B, Fersht A R. 2004. J Mol Biol 341: 1145-59

A1. Miroux, B. & Walker, J. E. (1996) J Mol Biol 260, 289-98.

A2. Packman, L. C., Borges, A. & Perham, R. N. (1988) Biochem J 252, 79-86.

A3. Dougherty, W. G., Carrington, J. C., Cary, S. M. & Parks, T. D. (1988) Embo J 7, 1281-7.

A4. Gill, S. C. & von Hippel, P. H. (1989) Anal Biochem 182, 319-26

A5. Goddard, T. D. & Kneller, D. G. SPARKY 3, University of California, San Francisco.

A6. Jackson, S. E. & Fersht, A. R. (1991) Biochemistry 30, 10436-10433.

A7. Friedler, A., Veprintsev, D. B., Freund, S. M., von Glos, K. I. & Fersht, A. R. (2005) Structure 13, 629-36.

A8. Friedler, A., Hansson, L. O., Veprintsev, D. B., Freund, S. M., Rippin, T. M., Nikolova, P. V., Proctor, M. R., Rudiger, S., & Fersht, A. R. (2002) Proc Natl Aced Sci U S A 99, 937-942.

B1. Joerger A C, Ang H C, Fersht A R. 2006. Proc Natl Acad Sci U S A 103: 15056-61

B2. Leslie AGW. 1992. Joint CCP4 and ESF-EACMB Newsletter on Protein Crystallography Vol. 26: Daresbury Laboratory, Warrington, UK

B3. Collaborative Computational Project N. 1994. Acta Crystallogr. D 50: 760-63

B4. Brütinger A T, Adams P D, Clore G M, DeLano W L, Gros P, et al. 1998. Acta Crystallogr. D 54: 905-21

B5. Turk D. 1992. Weiterentwicklung eines Programms für Molekülgrafik und Elektrondichte-Manipulation und seine Anwendung auf verschiedene Protein-Strukturaufklärungen. Technische Universität München

B6. Laskowski R A, MacArthur M W, Moss D S, Thornton J M. 1993. J. Appl. Crystallogr. 26: 283-91

1. A method for treating a subject who has a lesion or a tumour in which p53 carries a Y220C mutation, the method comprising administering to the subject a compound of formula (I): 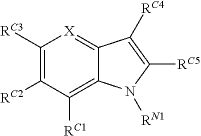
for use in a method of treatment of a subject who has a lesion or a tumour in which p53 carries a Y220C mutation
wherein X is selected from CR^{X} and N;
R^{N1} is selected from H and C₁-₄ alkyl, which may be substituted by SH or halo;
R^{C1} is selected from H and SH;
R^{C2} is selected from H and optionally substituted C₁₋₇ alkyl;
R^{C3} is selected from H and optionally substituted C₁-₇ alkyl;
R^{X} is selected from H, OH and NH₂;
R^{C4} is selected from:
(i) an optionally substituted C₃₋₁₂ N-containing heterocyclyl;
(ii) C(═O)NR^{N5}R^{N6}, where R^{N5} and R^{N6} are independently selected from H, optionally substituted C₁₋₇ alkyl, optionally substituted C₃₋₂₀ heterocyclyl and optionally substituted C₅₋₂₀ aryl or R^{N5} and R^{N6} and the nitrogen atom to which they are attached form an optionally substituted N-containing C₅₋₇ heterocyclyl group;
(iii) C(═O)OR^{O1}, where R^{O1} is selected from H, optionally substituted C₁₋₇ alkyl, optionally substituted C₃₋₂₀ heterocyclyl and optionally substituted C₅₋₂₀ aryl;
(iv) C(═O)NHNHSO₂R^{S1}, where R^{S1} is selected from H, optionally substituted C₁₋₇ alkyl, optionally substituted C₃₋₂₀ heterocyclyl and optionally substituted C₅₋₂₀ aryl;
(v) OC(═O)R^{C8}, where R^{C8} is selected from H, optionally substituted C₁₋₇ alkyl, optionally substituted C₃₋₂₀ heterocyclyl and optionally substituted C₅₋₂₀ aryl;
(vi) OC(═O)NR^{N7}R^{N8}, where R^{N7} and R^{N8} are independently selected from H, optionally substituted C₁₋₇ alkyl, optionally substituted C₃₋₂₀ heterocyclyl and optionally substituted C₅₋₂₀ aryl or R^{N7} and R^{N8} and the nitrogen atom to which they are attached form an optionally substituted N-containing C₅₋₇ heterocyclyl group; and
(vii) C(═O)CH₂NH₂, C(═O)NHNH₂, CHC(CN)₂, CHC(CN)C(═O)NH₂, and carboxy;
R^{C5} is selected from H, OH and NH₂;
or R^{C4} and R^{CS} together with the carbon atoms to which they are bound form an optionally substituted aromatic ring containing either 5 or 6 ring atoms, of formula: 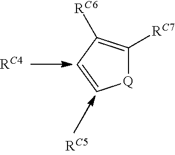
where Q represents O, N, or CRQ¹═CRQ², where RQ¹ and RQ² are independently selected from H, OH and NH₂;
R^{C6} is selected from H, OH and NH₂; and
R^{C7} is selected from optionally substituted C₃₋₁₂ N-containing heterocyclyl, NHC(═O)R^{C9}, CH₂NR^{N2}R^{N3} and NHC(═S)NHR^{N4}, _{where R}^{C9} is selected from optionally substituted C₁₋₇ alkyl, optionally substituted C₃-₂₀ heterocyclyl and optionally substituted C₅-_{20 aryl, R}^{N2} and R^{N3} are independently selected from H, optionally substituted C₁₋₇ alkyl, optionally substituted C₃₋₂₀ heterocyclyl and optionally substituted C₅₋₂₀ aryl or R^{N2} and R^{N3} and the nitrogen atom to which they are attached form an optionally substituted N-containing C₅₋₇ heterocyclyl group, and R^{N4} is selected from optionally substituted C₁₋₇ alkyl, optionally substituted C₃₋₂₀ heterocyclyl and optionally substituted C₅₋₂₀ aryl,
and when R^{C4} and R^{C5} are not bound together, R^{C3} may additionally be selected from OR^{O2}, where R^{O2} is a C₁₋₄ alkyl group, and C(═O)OR^{O3}, where R^{O3} is a C₁₋₄ alkyl group and R^{C2} may additionally be selected from halo.

2. A method according to claim 1, wherein R^{N1} is selected from H, ethyl, propyl and cyclopropyl.

3. A method according to claim 1, wherein R^{C1} is H.

4. A method according to claim 1, wherein R^{C2} is H.

5. A method according to claim 1, wherein R^{C2} is optionally substituted C₁₋₇ alkyl, where the optional substituents are selected from C₁₋₇ alkyl, C₃₋₇ heterocyclyl, C₅₋₇ aryl, halo, hydroxy, ether, nitro, cyano, acyl, carboxy, ester, amido, amino, acylamido, ureido, acyloxy and thiol.

6. A method according to claim 1, wherein R^{C3} is H.

7. A method according to claim 1, wherein R^{C3} is optionally substituted C₁₋₇ alkyl, where the optional substituents may be selected from C₁₋₇ alkyl, C₃₋₇ heterocyclyl, C₅₋₇ aryl, halo, hydroxy, ether, nitro, cyano, acyl, carboxy, ester, amido, amino, acylamido, ureido, acyloxy and thiol.

8. A method according to claim 1, wherein R^{X} is H.

9. A method according to claim 1, wherein R^{C4} is an optionally substituted C₃₋₁₂ N-containing heterocyclyl and R^{C5} is H.

10. A method according to of claim 1, wherein R^{C4} is C(═O)NR^{N5}R^{N6} and R^{C5} is H, and wherein:
(a) R^{N5} is selected from H and C₁₋₄ alkyl and R^{N6} is selected from: H, optionally substituted C₁₋₄ alkyl, where the optional substituents are selected from hydroxy, amino and C₅₋₉ aryl; and optionally substituted C₅₋₆ heterocyclyl, where the optional substituents are C₁₋₄ alkyl; or
(b) R^{N5} and R^{N6} and the nitrogen atom to which they are attached form optionally substituted piperidinyl or piperazinyl, where the optional substituents are selected from C₁₋₇ alkyl, hydroxy, C₅₋₇ heterocyclyl, C₅₋₇ aryl, amino and acyl.

11.-12. (canceled)

13. A method according to claim 1, wherein R^{C4} is C(═O)OR^{O1} and R^{C5} is H, and wherein R^{O1} is optionally substituted C₁₋₄ alkyl, where the optional substituents are selected from ether, oxyureido, C₅₋₆ aryl and amido.

14. (canceled)

15. A method according to claim 1, wherein R^{C4} is C(═O)NHNHSO₂R^{S1} and R^{C5} is H, and wherein R^{S1} is an optionally substituted C₅₋₆ aryl group, where the optional substituents are selected from alkoxy, ether and C₁₋₇ alkyl.

16. (canceled)

17. A method according to claim 1, wherein R^{C4} is OC(═O)NR^{N7}R^{N8} and R^{C5} is H, and wherein R^{N7} is H, and R^{N8} is optionally substituted C₅₋₆ aryl, where the optional substituents are selected from halo.

18. (canceled)

19. A method according to claim 1, wherein R^{C4} is OC(═O)R^{C8} and R^{C5} is H, and wherein R_{C8} is optionally substituted C₅₋₇ heterocyclyl.

20. (canceled)

21. A method according to claim 1, wherein R^{C4} is selected from C(═O)CH₂NH₂, C(═O)NHNH₂, CHC(CN)₂, CHC(CN)C(═O)NH₂, and carboxy and R^{C5} is H.

22. A method according to claim 1, wherein R^{C4} and R^{C5} together with the carbon atoms to which they are bound form an optionally substituted aromatic ring containing either 5 or 6 ring atoms, of formula: 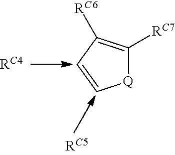
where Q is CR^{Q1}═CR^{Q2}, where R^{Q1} is selected from H and OH, and R^{Q2} is H;
R^{C6} is selected from H, OH and NH₂; and
R^{C7} is selected from optionally substituted C₃₋₁₂ N-containing heterocyclyl, NHC(═O)R^{C9}, CH₂NR^{N2}R^{N3} _{and NHCl (═S)}_{NHR}^{N4}.

23. A method according to claim 22, wherein R^{C6} is H.

24. A method according to claim 22, wherein R^{C7} is an optionally substituted C₃₋₁₂ N-containing heterocyclyl.

25. A method according to claim 22, wherein R^{C7} is NHC(═O)R^{C9}, where R^{C9} is selected from:
(a) optionally substituted C₁₋₄ alkyl group, where the optional substituents are selected from acyloxy, C₅₋₇ aryl, amino, thioester and C₃₋₇ heterocyclyl; and
(b) optionally substituted C₅₋₆ aryl group, where the optional substituents may be selected from C₁₋₇, alkyl, C heterocyclyl, C₅₋₇ aryl, halo, hydroxy, ether, nitro, cyano, acyl, carboxy, ester, amido, amino, acylamido, ureido, acyloxy and thiol.

26.-27. (canceled)

28. A method according to claim 22, wherein R^{C7} is CH₂NR^{N2}R^{N3}, where R^{N2} and R^{N3} are independently selected from H, optionally substituted C₁₋₇ alkyl, optionally substituted C₃₋₂₀ heterocyclyl and optionally substituted C₅₋₂₀ aryl or R^{N2} and R^{N3} and the nitrogen atom to which they are attached form an optionally substituted N-containing C₅₋₇ heterocyclyl group.

29. A method according to claim 22, wherein R^{C7} is NHC(═S)NHR^{N4}, where R^{N4} is selected from optionally substituted C₁₋₇ alkyl, optionally substituted C₃₋₂₀ heterocyclyl and optionally substituted C₅₋₂₀ aryl.

30. A method for treating a cell in which p53 carries a Y220C mutation, the method comprising contacting the cell with a compound of formula (I) as defined in claim.

31. (canceled)

32. A method for stabilizing a p53 protein which carries a Y220C mutation, the method comprising bringing the p53 into contact with a compound of formula (I) as defined in claim 1.

33. A method of determining the binding of a molecule to a p53 which carries a Y220C mutation, the method comprising bringing the molecule into contact with said p53 in competition with a compound of formula (I) as defined in claim 1, and measuring the binding or displacement of one or other of said compounds.

34. A method according to claim 33, wherein one or both of the compounds carries a label, such as a radiolabel, chromophore, fluorophore or a fluorine function for competition-based ¹⁹F-screening using magnetic resonance techniques.

35-36. (canceled)
